(19) United States
(12) Patent Application Publication
(10) Pub. No.: US 2005/0267128 A1
(43) Pub. Date: Dec. 1, 2005

(54) Compounds, compositions and methods of modulating the mitochondrial apoptosis-induced channel (MAC)

(76) Inventors: Kathleen W. Kinnally, Albany, NY (US); Evgeny Pavlov, Calgary (CA)

Correspondence Address: KLAUBER & JACKSON, 411 HACKENSACK AVENUE, HACKENSACK, NJ, 07601, US

(21) Appl. No.: 11/137,119

(22) Filed: May 25, 2005

Related U.S. Application Data
(60) Provisional application No. 60/574,183, filed on May 25, 2004.

(51) Int. Cl. A61K 31/495; A61K 31/138; A61K 31/24

(52) U.S. Cl. 514/255.03; 514/651; 514/534

(57) ABSTRACT

The present invention relates to methods of treating conditions or diseases in which apoptosis is either desirable or undesirable. In particular, in conditions whereby it is desirable to inhibit unwanted cellular proliferation such as in cancers or in hyperproliferative disorders, treatment with an agent that promotes opening of the mitochondrial apoptosis induced channel (MAC) is beneficial. In conditions whereby there is unwanted apoptosis, such as in certain conditions exemplified by cell death at the site of injury, such as stroke, Alzheimer's disease, myocardial infarct, traumatic brain injury and in spinal cord injury, it is desirable to treat with an agent that prevents opening of the MAC, or promotes closure of the MAC. The present invention also relates to pharmaceutical compositions comprising such agents. Methods of screening for novel agents are also disclosed.

### GOVERNMENT RIGHTS CLAUSE

The research leading to the present invention was supported by NIH grant number GM57249. Accordingly, the Government has certain rights in the invention.

### FIELD OF THE INVENTION

This invention relates to the mitochondrial apoptosis-induced channel (MAC) and methods and compounds that effectuate induction of apoptosis or inhibition of apoptosis via this channel. Furthermore, particular compounds have been identified that may prove beneficial in treating conditions characterized in part by the presence of apoptotic cell death wherein such apoptotic cell death is undesirable or alternatively, in conditions that are characterized by undesirable cellular proliferation wherein apoptotic cell death is desirable.

### BACKGROUND OF THE INVENTION

Apoptosis is a phenomenon fundamental to higher eukaryotes and essential to mechanisms controlling tissue homeostasis. Accordingly, apoptosis is integral to such diverse cellular processes as tissue remodeling and organogenesis, as well as in chemotherapy-induced tumor regression. Morphological indicia of apoptosis include membrane blebbing, chromatin condensation and fragmentation, and formation of apoptotic bodies. Degradation of genomic DNA during apoptosis results in formation of characteristic, nucleosome sized DNA fragments; this degradation produces a diagnostic (about) 180 bp laddering pattern when analyzed by gel electrophoresis. A later step in the apoptotic process is degradation of the plasma membrane, rendering apoptotic cells leaky to various dyes (e.g., trypan blue and propidium iodide). A key early event in the apoptotic cascade in many cell types is the release of cytochrome c. Once in the cytoplasm, cytochrome c and procaspase 9 bind the cytoplasmic protein apaf-1 and dATP to form apoptosomes that promote caspase activation and destruction of the cell (Wang, X. (2001), Genes & Development. 15, 2922-33; Liu, X., Kim, C. N., Yang, J., Jemmerson, R. and Wang, X. (1996), Cell 86, 147-157). Recently, a new set of proteins has been identified that further regulate apoptosome formation (Jiang, X., Kim, H.-E., Shu, H., Zhao, Y., Zhang, H., Kofron, J., Donnelly, J., Burns, D., Ng, S.-c., Rosenberg, S. and Wang, X. (2003), Science 299, 223-226). The mechanisms by which pro-apoptotic factors are released from mitochondria early in apoptosis are not well understood. It has been speculated that a permeability transition pore (PTP^{a}) of the inner membrane opens and causes swelling of the matrix space. As the inner membrane has a much greater surface area than the outer membrane, the ensuing swelling ruptures the outer membrane and spills cytochrome c and other pro-apoptotic proteins into the cytoplasm. However, cytochrome c release has also been shown to occur in the absence of mitochondrial depolarization and a loss of outer membrane integrity in some cell types.

In addition to its involvement in overall cell population homeostasis, apoptosis also plays a substantial role in cell death that occurs in conjunction with various disease and injury conditions. For example, apoptosis is involved in the neuronal damage caused by neurodegenerative disorders, including Alzheimer's disease (Barinaga, Science 281:1303-1304), Huntington's disease, spinal-muscular atrophy, stroke (reviewed in Rubin, British Med. Bulle., 53(3):617-631, 1997; and Barinaga, Science 281:1302-1303), and transient ischemic neuronal injury, as in spinal cord injury. Accordingly, it would be of great benefit to prevent undesired apoptosis in these various diseases and injury situations.

Furthermore, in conditions such as cancer and other hyperproliferative cell conditions, it would be advantageous to be able to enhance apoptosis to stop unregulated or undesired growth.

The mitochondrial apoptosis-induced channel, MAC, is induced early in apoptosis (Pavlov, E. V., Priault, M., Pietkiewicz, D., Cheng, E. H., Antonsson, B., Manon, S., Korsmeyer, S. J., Mannella, C. A. and Kinnally, K. W. (2001), J Cell Biol 155, 725-31). The high conductance (2-6 nS) suggests MAC has a pore that is >4 nm in diameter. Evidence is mounting that MAC provides the pathway through the outer membrane for release of the 3.3 nm diameter cytochrome c. Not only does cytochrome c reduce the conductance of MAC in a manner consistent with its partitioning into the pore of MAC (Guo, L., Pietkiewicz, D., Pavlov, E. V., Grigoriev, S. M., Kasianowicz, J. J., Dejean, L. M., Korsmeyer, S. J., Antonsson, B. and Kinnally, K. W. (2004), Am J Physiol Cell Physiol 286, C1109-17), but proteoliposomes expressing MAC activity fail to retain cytochrome c. MAC activity is present in multiple different cell types (CSM14.1, and various clones of FL5.12 and HeLa cells) during cytochrome c release.

The potential for identifying compounds that affect the pharmacological utility of this channel for modulating cell death or inhibition of cell death has not been studied. It is with respect to this aspect that the present application is directed.

### SUMMARY OF THE INVENTION

The mitochondrial apoptosis induced channel (MAC) is a channel for which no pharmacological profile has been identified. It is with respect to the identification of methods, compounds and compositions that modulate the opening or closing of this channel, and allowing, or alternatively preventing, the release of cytochrome c through this channel, which in turn plays a role in induction of a death signal to neighboring cells through gap junctions that the present application is directed.

Accordingly, a first aspect of the invention provides a method of inducing apoptosis in cells in vitro or in vivo comprising administering an agent that promotes opening of the mitochondrial apoptosis induced channel (MAC), wherein said opening results in release of cytochrome c and subsequent release of a death signal. In a particular embodiment, the death signal is other than cytochrome c, that is, a small molecule other than cytochrome c that is capable of traversing gap junctions. In another particular embodiment, the MAC channel is integral to the bystander effect in vivo and the release of a death signal results in apoptosis of cells outside of the area of the immediate cellular or tissue insult.

A second aspect of the invention provides a method of treating a disease or condition characterized in part by the presence of apoptotic cell death, wherein said apoptotic cell death is undesirable, comprising administering an agent that prevents opening of the mitochondrial apoptosis-induced channel (MAC), or promotes closure of the mitochondrial apoptosis-induced channel (MAC). In a particular embodiment, the disease or condition is selected from the group consisting of stroke, myocardial infarction, Alzheimer's disease, traumatic brain injury, spinal cord injury, AIDS and any other medical condition characterized in part by the presence of unwanted or undesirable apoptotic cell death. In another particular embodiment, the agent is selected from the group consisting of trifluoperazine, dibucaine and propranolol.

A third aspect of the invention provides a method of treating a disease or condition wherein said disease or condition is characterized by unwanted or undesirable cellular proliferation, comprising administering an agent that promotes opening of the mitochondrial apoptosis-induced channel (MAC) and apoptosis. In a particular embodiment, the method results in release of cytochrome c and subsequent release of a death signal. In a particular embodiment, the death signal is not cytochrome c, but is a small molecule capable of traversing gap junctions. In yet another embodiment, the modulation of MAC to initiate the release of cytochrome c and a small molecule other than cytochrome c results in a death signal to the neighboring cells which lie outside of the immediate area of the initial insult or injury (the “bystander effect”) through gap junctions. In yet another particular embodiment, the disease or condition is selected from the group consisting of a cancer and any other hyperproliferative disorder for which inhibition of cellular proliferation and cell death is desirable. In yet another particular embodiment, cell death is achieved by apoptosis.

A fourth aspect of the invention provides for pharmaceutical compositions comprising an agent that modulates MAC, that is, either promotes opening of MAC or promotes closing of MAC or that inhibits the opening of MAC, and a pharmaceutically acceptable carrier.

A fifth aspect of the invention provides a method of screening for novel compounds or modulators that effectuate the closing or the opening of the mitochondrial apoptosis induced channel. In a particular embodiment, the method of screening provides for a patch clamp technique for identification of such compounds. In yet another particular embodiment, the method provides for monitoring the efflux of cytochrome c from either cells expressing endogenous MAC or cells that have been genetically engineered to express MAC or the functional components thereof. In yet another particular embodiment, the cells may be selected from the group consisting of MG63, CSM14.1, various clones of FL5.12 and HeLa cells Mitochondrial outer membranes isolated from untreated (with MAC components that are not assembled) or apoptotic cells (with MAC fully formed) will be fused with liposomes and these liposomes will be used to trap fluorescently labeled (eg. FITC) cytochrome c or a similarly sized molecule (eg. Dextran or Polyethylene glycol 10 kDa MW). Alternatively, when looking for an opener, mitochondria can be isolated from untreated cells, drug may be added, and the release of cytochrome c can be monitored by ELISA. These systems would then be used together. For example, an opener of MAC can be added to isolated mitochondria that is blocked by a compound which is an inhibitor. In yet another particular embodiment, the method for identifying modulators of a mitochondrial apoptosis induced channel (MAC) comprises contacting a test compound with the MAC and detecting the activity of the MAC. If the MAC activity is inhibited, a modulator useful for treatment of a disease or condition selected from the group consisting of stroke, Alzheimer's disease, myocardial infarction, traumatic brain injury and spinal cord injury has been identified. If the MAC activity is enhanced, a modulator useful for treatment of a disease or condition selected from the group consisting of a cancer and any hyperproliferative disorder for which induction of apoptosis is desirable has been identified.

Other objects and advantages will become apparent from a review of the ensuing detailed description and attendant claims taken in conjunction with the following illustrative drawings. All references cited in the present application are incorporated herein in their entirety.

### BRIEF DESCRIPTION OF THE DRAWINGS

FIG. 1. Fast blockade of MAC by dibucaine. A. Immunoblots show the presence of the outer membrane protein VDAC but not the inner membrane protein cytochrome oxidase subunit IV (CoxIV) in the outer membranes (OM, 2 μg) purified from mitochondria of apoptotic FL5.12 cells. Inner membranes (IM, 2 μg) are the positive control for CoxIV. B. Representative current trace of a MAC recorded at +20 mV with 2 kHz filtration is shown 10 seconds after perfusion of the bath with 50 μM Dibucaine. O and C indicate open and closed conductance states. C. The dibucaine induced closure of MAC is reversible. Current traces (right) and total amplitude histograms (left) of MAC are shown before and after perfusion with media containing 200 μM dibucaine, and after perfusion with 150 mM KCl 5 mM HEPES to wash out dibucaine. O and C correspond to open and closed conductance states.

FIG. 2. Propranolol and trifluoperazine block the conductance of MAC. Current traces (right) and total amplitude histograms (left) of two patches in which MAC was recorded at −30 mV before and after application of (A) 200 μM propranolol and (B) 10 μM trifluoperazine (TFP). C and O indicate closed and open conductance states.

FIG. 3. Inhibitory effects of Dibucaine, Propanolol and Trifluoperazine. % Inhibition of conductance (% mean conductance with/without drug) is plotted as a function of the log concentration (M) of dibucaine, propranolol and trifluoperazine (A), and lidocaine and cyclosporine A (B). The data are best fit with lines for dibucaine, propranolol and trifluoperazine with IC₅₀ of 39, 52 and 0.9 μM and correlation coefficients (R²) of 0.99, 0.96 and 0.95, respectively. The correlation coefficients (R²) for the best fits for lidocaine and cyclosporine A are 0.92 and 0.90, respectively.

FIG. 4. Immunoprecipitation of Bax depletes MAC activity from a partially purified fraction of mitochondria of apoptotic cells. (A) Western blot shows 0.2 μg Bax antibodies immunoprecipitates 2 ng Bax from fraction 23 but not fraction 38 purified from apoptotic HeLa cells (Antonsson, B., Montessuit, S., Sanchez, B. and Martinou, J. C. (2001).Bax is present as a high molecular weight oligomer/complex in the mitochondrial membrane of apoptotic cells. J. Biological Chemistry 276, 11615-11623). This polyclonal Bax antibody was raised against Bax's N-terminus (Santa Cruz), which is not normally exposed in monomeric Bax. Fraction 38 elutes just after 25 kDa MW marker and contains monomeric Bax. Fraction 23 elutes near the 232 MW marker and is oligomeric Bax with the N-terminus exposed, i.e., activated Bax. Fractions were immunoprecipitated with equivalent total rabbit IgG (control Ab) as a control. The immunoprecipitated pellets (P) and their supernatants (S) were subjected to SDS-PAGE, and the presence of Bax was assessed by Western blot. (B) Apoptotic fraction 23 (Apoptotic Fx23) contains MAC activity that is depleted from the supernatants by immunoprecipitation by Bax but not by control IgG. Little MAC activity is present in apoptotic fraction 38 and control fractions 23 & 38. n=20-23 independent patches/point. (C) Taken from ref. 6, western blots show the presence of Bax and Bak, but not VDAC or ANT in fraction 23, and Bax but not Bak, VDAC or ANT in fraction 38. Arrows indicate fractions 23 and 38.

FIG. 5. Bax immunoprecipitation depletes MAC activity. A, B. Whole mitochondrial lysates of staurosporine-treated (apoptotic) and untreated (control) HeLa cells containing 40 ng Bax (34 μg and 50 μg protein, respectively) were immunoprecipitated with antibodies against Bax or total rabbit IgG (control). Supernatants (S) and pellets (P) were assayed by western blot for Bax content. Supernatants were dialyzed and reconstituted into proteoliposomes so that MAC could be detected by patch clamp techniques. A. Western blots show Bax levels in the supernatants and pellets after immunoprecipitation of apoptotic and control lysates. B. MAC detection frequency was determined before and after immunoprecipitation of the same fractions with anti-Bax and control antibodies. Immunoprecipitation with anti-Bax antibodies resulted in ˜10-fold decrease in MAC detection. These anti-Bax antibodies recognize activated Bax under “native” conditions. N is the number of independent patches tested. C. Partially purified fraction 23 of the apoptotic lysates were immunoprecipitated with anti-Bax and control antibodies (IgG). Supernatants and pellets were probed for the presence of Bax, Bak, VDAC, and t-Bid by western as shown. While present, Bak was not immunoprecipitated by anti-Bax antibodies in control fraction 23 (not shown).

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

### DEFINITIONS

The terms used herein have the meanings recognized and known to those of skill in the art, however, for convenience and completeness, particular terms and their meanings are set forth below.

“Agent” refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

“Hyperproliferative Disorders” refers to diseases that result from the abnormal growth of cells. These can include cancers, pre-malignant states as well as inflammatory states such as rheumatoid arthritis or abnormal proliferation of cells in other tissues of the human body, such as psoriasis.

“Analog” as used herein, refers to a chemical compound, a nucleotide, a protein, or a polypeptide that possesses similar or identical activity or function(s) as the chemical compounds, nucleotides, proteins or polypeptides having the desired activity and therapeutic effect of the present invention (eg. to inhibit cellular proliferation for treatment of mammals having cancer or hyperproliferative disorders or to treat mammals suffering from conditions wherein cell death by apoptosis results in further damage to surrounding tissues, such as in stroke, Alzheimer's disease, traumatic brain injury and spinal cord injury), but need not necessarily comprise a compound that is similar or identical to those compounds of the preferred embodiment, or possess a structure that is similar or identical to the agents of the present invention.

“Derivative” refers to chemically synthesized organic molecules that are functionally equivalent to the active parent compound, but may be structurally different. It may also refer to chemically similar compounds which have been chemically altered to increase bioavailability, absorption, or to decrease toxicity.

A “therapeutically effective amount” is an amount sufficient to decrease or prevent the symptoms associated with the conditions disclosed herein, including cancer, hyperproliferative disorders, stroke, Alzheimer's disease, traumatic brain injury, spinal cord injury, myocardial infarction or other related conditions contemplated for therapy with the compositions of the present invention.

“Treatment” or “treating” refers to therapy, prevention and prophylaxis and particularly refers to the administration of medicine or the performance of medical procedures with respect to a patient, for either prophylaxis (prevention) or to cure or reduce the extent of or likelihood of occurrence of the infirmity or malady or condition or event in the instance where the patient is afflicted.

“Apoptosis” refers to programmed cell death and is characterized by membrane blebbing, chromatin condensation and fragmentation, and formation of apoptotic bodies. Degradation of genomic DNA during apoptosis results in formation of characteristic, nucleosome sized DNA fragments; this degradation produces a diagnostic (about) 180 bp laddering pattern when analyzed by gel electrophoresis. A later step in the apoptotic process is degradation of the plasma membrane, rendering apoptotic cells leaky to various dyes (e.g., trypan blue and propidium iodide). Specific markers for apoptosis include, but are not limited to, annexin V staining, DNA laddering, staining with dUTP and terminal transferase [TUNEL].

“Small molecule” or “small organic molecule” is an organic compound (or organic compound complexed with an inorganic compound (e.g., metal)) that has a molecular weight of less than 3 kilodaltons, and preferably less than 1.5 kilodalton. A “small molecule other than cytochrome c capable of traversing gap junctions” refers to a molecule preferably below 1000 daltons that can cross or traverse between cells in a passive fashion. Examples of these molecules include, but are not limited to Ca++, c-AMP, glutathione, amino acids, sugars and nucleotides, free radicals, reactive oxygen species (ROS), reactive nitrogen species (RNS), and nitric oxide (NO).

“Subject” or “patient” refers to a mammal, preferably a human, in need of treatment for a condition, disorder or disease.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

“Death signal” refers to the trigger that ultimately leads to cell death by apoptosis and also plays a role in the induction of the bystander effect as described below. Apoptotic death can be triggered by a wide variety of stimuli, and not all cells necessarily will die in response to the same stimulus. Among the more studied death stimuli is DNA damage (by irradiation or drugs used for cancer chemotherapy), which in many cells leads to apoptotic death via a pathway dependent on p53. Some hormones such as corticosteroids lead to death in particular cells (e.g., thymocytes), although other cell types may be stimulated. Some cells types express Fas, a surface protein which initiates an intracellular death signal in response to crosslinking. In other cases cells appear to have a default death pathway which must be actively blocked by a survival factor in order to allow cell survival. When the survival factor is removed, the default apoptotic death program is triggered. The mechanism underlying the small molecule death signal induced apoptosis is unknown at this time. Some likely candidates for the death signal are calcium, P3, cAMP, cGMP, ROS, and NO.

“Bystander effect” as used herein refers to the death of cells outside of the immediate area of cells or tissue that suffered an initial insult or injury. For example, it has been observed that cells nearby irradiated cells die during radiation therapy. It is likely that this process is fundamental to tissue homeostasis whereby injured cells notify their neighbors of the nature of an insult that could endanger tissue homeostasis. Surprisingly, little is known or understood of the fundamental mechanisms regulating the bystander effect. It is likely that gap junctions are essential to this process of communicating a death signal, as expression of different connexins modifies the bystander effect in other cell types. However, there is little information regarding the nature of the death signal for the bystander effect and its means of propagation. However, the release of cytochrome c generates a death signal that can be propagated to the mitochondria of other cells. Several cell types including cardiac, epithelial, and osteoblast cells rely upon gap junction communication for tissue homoeostasis, and normal cell function. Interestingly, some, but not all, cell types that “share” Lucifer yellow staining are susceptible to apoptosis if their nearest neighbor is microinjected with cytochrome c or calcium. A single cell is microinjected with a lethal dose of cytochrome c and rapidly enters apoptosis. Within several hours, several of the other cells die as they bleb and their nuclei condense. These cells are coupled as indicated by the fluorescence of several cells when a single cell is microinjected with Lucifer yellow, a dye used to show communication through gap junctions.

The “mitochondrial apoptosis-induced channel” or “MAC” is a high conductance voltage independent channel that is found in every cell in the body that has mitochondria. This channel forms in the outer membrane of mitochondria early in apoptosis before the onset of other apoptotic markers, such as Annexin-V labeling (Guo, L. et al. (2004), Am. J. Physiol. 286: C1109-C1117). This channel has a pore size of between 3.5 to 10 nm and is found in many cell types.

“Inhibitors,” “activators”, “agents that promote opening”, “agonists”, or “openers,” “agents that promote closing” or “agents that prevent opening”, or “modulators” or “regulators” of mitochondrial apoptosis-induced channel (MAC) refer to inhibitory or activating molecules identified using in vitro and in vivo assays for mitochondrial apoptosis-induced channel function. In particular, inhibitors, and agents that promote closing or agents that prevent opening refer to compounds that decrease mitochondrial apoptosis-induced channel function, including release of cytochrome c and a subsequent death signal to cells surrounding the immediate area of insult or injury, thereby reducing further cell death in a subject at the site of injury. Inhibitors are also compounds that decrease, block, prevent, delay activation, inactivate, desensitize, or down regulate the channel, or speed or enhance deactivation. Activators, or agents that promote opening are compounds that open, activate, facilitate, enhance activation, sensitize or up regulate channel activity, or delay or slow inactivation. Such agents are compounds or molecules that are effective at promoting cellular apoptosis. Such assays for inhibitors and activators also include, e.g., expressing recombinant MAC, or the components of MAC in cells or cell membranes, or in liposomes and then measuring flux of cytochrome c through the channel directly or indirectly. Alternatively, cells expressing endogenous MAC channels can be used in such assays (e.g., MG63 osteoblasts, MEFs, MAFs, CSM14.1, and various clones of FL5.12 leukemia cells and HeLa cells).

General Description

Apoptosis is a phenomenon fundamental to higher eukaryotes and essential to the mechanisms underlying tissue homeostasis. The release of cytochrome c from mitochondria is considered the commitment step of apoptosis in many cell types and is tightly regulated by Bcl-2 family proteins (Liu, X., Kim, C. N., Yang, J., Jemmerson, R. and Wang, X. (1996) Cell 86, 147-157; Kluck, R. M., Bossy-Wetzel, E., Green, D. R. and Newmeyer, D. D. (1997) Science 275, 1132-1136; Yang, J., Liu, X., Bhalla, K., Kim, C. N., Ibrado, A. M., Cai, J., Peng, T. I., Jones, D. P. and Wang, X. (1997) Science 275, 1129-32; Wei, M. C., Zong, W. X., Cheng, E. H., Lindsten, T., Panoutsakopoulou, V., Ross, A. J., Roth, K. A., MacGregor, G. R., Thompson, C. B. and Korsmeyer, S. J. (2001) Science 292, 727-730.) While the permeability transition pore is implicated in cytochrome c release in some systems (Kroemer, G. and Reed, J. C. (2000) Nat. Med. 6, 513-19; Brenner, C. and Kroemer, G. (2000) Science 289, 1150-51), recent investigations show cytochrome c can exit directly through a pore in the mitochondrial outer membrane without loss of outer membrane integrity (Brenner, C. and Kroemer, G. (2000) Science 289, 1150-51; Guo, L., Pietkiewicz, D., Pavlov, E. V., Kasianowicz, J. J., Korsmeyer, S. J., Antonsson, B. and Kinnally, K. W. (2004) American Journal of Physiology, Cell Biology. 286, C1109-17; Antonsson, B., Conti, F., Ciavatta, A., Montessuit, S., Lewis, S., Martinou, I., Bernasconi, L., Bernard, A., Mermod, J. J., Mazzei, G., Maundrell, K., Gambale, F., Sadoul, R. and Martinou, J. C. (1997) Science 277, 370-372; Martinou, J. C. and Green, D. R. (2001) Nature Reviews Molecular Cell Biology 2, 63-66; Saito, M., Korsmeyer, S. J. and Schlesinger, P. H. (2000) Nat Cell Biol 2, 553-5; Shimizu, S., Matsuoka, Y., Shinohara, Y., Yoneda, Y. and Tsujimoto, Y. (2001) J. Cell Biol. 152, 237-250; De Giorgi, F., Lartigue, L., Bauer, M. K., Schubert, A., Grimm, S., Hanson, G. T., Remington, S. J., Youle, R. J. and Ichas, F. (2002) FASEB Journal. 16, 607-9; Pavlov, E. V., Priault, M., Pietkiewicz, D., Cheng, E.H.-Y., Antonsson, B., Manon, S., Korsmeyer, S. J., Mannella, C. A. and Kinnally, K. W. (2001) J. Cell Biology 155, 725-732).

A high conductance channel forms in the mitochondrial outer membrane early in apoptosis before the onset of other apoptotic markers, e.g., Annexin-V labeling (Guo, L., Pietkiewicz, D., Pavlov, E. V., Kasianowicz, J. J., Korsmeyer, S. J., Antonsson, B. and Kinnally, K. W. (2004) American Journal of Physiology, Cell Biology. 286, C1109-17). The appearance of this mitochondrial apoptosis-induced channel, or MAC, is prevented by overexpression of Bcl-2 (Pavlov, E. V., Priault, M., Pietkiewicz, D., Cheng, E.H.-Y., Antonsson, B., Manon, S., Korsmeyer, S. J., Mannella, C. A. and Kinnally, K. W. (2001) J. Cell Biology 155, 725-732). Furthermore, the single channel activity of MAC is modified by physiological levels of cytochrome c (Guo, L., Pietkiewicz, D., Pavlov, E. V., Kasianowicz, J. J., Korsmeyer, S. J., Antonsson, B. and Kinnally, K. W. (2004) American Journal of Physiology, Cell Biology. 286, C1109-17). These findings support a role for MAC in the release of cytochrome c and possibly other factors early in apoptosis.

The mitochondrial apoptosis-induced channel (MAC) is a channel with a pore size of between 3.5 nm to at least 10 nm, which forms in the outer membrane of mitochondria early in apoptosis. This channel is slightly cation selective but not voltage dependent. BAX, a Bcl-2 family protein with pro-apoptotic action, is a component of MAC. The anti-apoptotic protein bcl-2 prevents the detection of MAC, presumably because the association of bcl-2 with BAX prevents MAC formation. The inventors provide herein a means of modulating the mitochondrial apoptosis induced channel (MAC) such that diseases or conditions in which apoptosis of cells is desirable (such as in cancer or hyperproliferative disorders) may be treated by agents that open the MAC, or alternatively, diseases or conditions in which apoptosis of cells is undesirable (stroke, myocardial infarct, Alzheimer's disease, traumatic brain injury, spinal cord injury), may be treated by agents that promote closing of the MAC, or by agents that prevent opening of the MAC. Accordingly, the inventors have identified several agents that modulate the function of MAC. In particular, dibucaine induces a fast blockade of MAC with an IC₅₀ of 39 μM. In contrast, the IC₅₀ for propranolol and trifluoperazine are 52 μM and 0.9 μM, respectively, and these drugs likely destabilize the open state of MAC. These agents, and others to be identified by the methods described herein, should be valuable tools in the study of apoptosis. Furthermore, the studies provided herein indicate that MAC may be a potential target for identifying other agents useful for treatment of conditions whereby apoptotic cell death is desirable (ie. cancer and hyperproliferative diseases) or in treating conditions wherein cellular apoptosis is undesirable, such as in stroke, myocardial infarction, Alzheimer's disease, or spinal cord injury or traumatic brain injury. Accordingly, profiling MAC's pharmacology may generate novel therapeutic regimens for disease.

The inventors have recordings of MAC made by patch clamping mitochondria within apoptotic cells. These patch-clamp studies were the first direct demonstration of the existence of MAC inside cells. The preliminary data are consistent with the notion that MAC provides a pathway for cytochrome c to exit the mitochondria and that anti-apoptotic Bcl-2 completely suppresses this channel activity.

The mechanisms underlying the regulation of MAC by Bcl-2 family proteins are not known. Nevertheless, as evidenced by the studies performed herein, whereby the inventors have demonstrated that certain amphiphilic cations are inhibitors of MAC, this channel has been identified as a potential therapeutic target for both cancer and degenerative diseases.

Accordingly, one aspect of the invention provides a method of inducing apoptosis in cells in vitro or in vivo comprising administering an agent that promotes opening of the mitochondrial apoptosis induced channel (MAC). Such agents may be small organic molecules, peptides, lipids, lipoproteins, or nucleic acid molecules. The opening of this channel results in release of cytochrome c and subsequent release of a death signal. In a particular embodiment, the death signal is other than cytochrome c, that is a small molecule other than cytochrome c capable of traversing gap junctions. In another particular embodiment, the MAC channel is integral to the bystander effect in vivo and the release of a death signal results in apoptosis of cells outside of the area of the immediate cellular or tissue insult.

Another aspect of the invention provides methods of treating specific diseases or conditions wherein apoptotic cell death is either desirable or is not desirable. For example, one particular embodiment provides a method of treating a disease or condition characterized in part by the presence of apoptotic cell death, wherein said apoptotic cell death is undesirable, comprising administering an agent that prevents opening of the mitochondrial apoptosis-induced channel (MAC), or promotes closure of the mitochondrial apoptosis-induced channel (MAC). In a particular embodiment, the disease or condition is selected from the group consisting of stroke, myocardial infarction, Alzheimer's disease, traumatic brain injury, spinal cord injury, AIDS and any other medical condition characterized in part by the presence of unwanted or undesirable apoptotic cell death. In another particular embodiment, the agent is selected from the group consisting of trifluoperazine, dibucaine and propranolol.

Another particular embodiment of the invention provides a method of treating a disease or condition wherein said disease or condition is characterized by unwanted or undesirable cellular proliferation, comprising administering an agent that promotes opening of the mitochondrial apoptosis-induced channel (MAC) and apoptosis. In a particular embodiment, the method results in release of cytochrome c and subsequent release of a death signal. In a particular embodiment, the death signal is not cytochrome c, but is a small molecule capable of traversing gap junctions. In yet another embodiment, the modulation of MAC to initiate the release of cytochrome c and a small molecule other than cytochrome c results in a death signal to the neighboring cells which lie outside of the immediate area of the initial insult or injury (the “bystander effect”) through gap junctions. In yet another particular embodiment, the disease or condition is selected from the group consisting of a cancer and any other hyperproliferative disorder for which inhibition of cellular proliferation and cell death is desirable. In yet another particular embodiment, cell death is achieved by apoptosis.

Another aspect of the invention provides for pharmaceutical compositions comprising an agent that modulates MAC, that is, either promotes opening of MAC or promotes closing of MAC or that inhibits the opening of MAC, and a pharmaceutically acceptable carrier.

Another aspect of the invention provides a method of screening for novel compounds or modulators that effectuate the closing or the opening of the mitochondrial apoptosis induced channel. In a particular embodiment, the method of screening provides for a patch clamp technique for identification of such compounds. In yet another particular embodiment, the method provides for monitoring the efflux of cytochrome c from either cells expressing endogenous MAC or cells that have been genetically engineered to express MAC or the functional components thereof. In yet another particular embodiment, the cells may be selected from the group consisting of MG63 osteoblasts, MEFs, MAFs, CSM14.1, various clones of FL5.12 and HeLa cells. Mitochondrial outer membranes isolated from untreated (with MAC components that are not assembled) or apoptotic cells (with MAC fully formed) will be fused with liposomes and can be used to trap fluorescently labeled (eg. FITC) cytochrome c or a similarly sized molecule (eg. Dextran or Polyethylene glycol 10 kDa MW). Alternatively, when looking for an opener, mitochondria can be isolated from untreated cells, drug can be added, and the release of cytochrome c can be monitored by ELISA. These systems would then be used together. For example, an opener can be added to isolated mitochondria that is then blocked by an inhibitor.

Therapeutic and Prophylactic Compositions and Their Use

The invention provides methods of treatment comprising administering to a subject an effective amount of an agent of the invention. In a preferred aspect, the compound is substantially purified (e.g., substantially free from substances that limit its effect or produce undesired side-effects). The subject is preferably an animal, including but not limited to animals such as monkeys, cows, pigs, horses, chickens, cats, dogs, etc., and is preferably a mammal, and most preferably human. In one specific embodiment, a non-human mammal is the subject. In another specific embodiment, a human mammal is the subject.

Various delivery systems are known and can be used to administer a compound of the invention, e.g., encapsulation in liposomes, microparticles, or microcapsules. Methods of introduction can be enteral or parenteral and include but are not limited to intradermal, intramuscular, intraperitoneal, intravenous, subcutaneous, intranasal, epidural, topical and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. In a specific embodiment, it may be desirable to administer the pharmaceutical compositions of the invention locally to the area in need of treatment.

Another aspect of the invention provides for pharmaceutical compositions comprising purified agents that promote opening of the MAC for therapeutic use in the treatment of cancer or hyperproliferative diseases or conditions, or alternatively agents that promote closing of the MAC for treatment of conditions in which apoptotic cell death is undesirable, such as but not limited to, stroke, Alzheimer's disease, myocardial infarction, traumatic brain injury and spinal cord injury.

One embodiment features treatment of a wide range of cancers or hyperproliferative conditions with pharmaceutical compositions containing acceptable carriers and excipients. Thus, compositions and methods provided herein are particularly deemed useful for the treatment of hyperproliferative disorders including solid tumors such as skin, breast, brain, cervical carcinomas, testicular carcinomas, etc. More particularly, cancers that may be treated by the compositions and methods of the invention include, but are not limited to, Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma [squamous cell, undifferentiated small cell, undifferentiated large cell, adenocarcinoma], alveolar [bronchiolar] carcinoma, bronchial adenoma, sarcoma, lymphoma, chondromatous hamartoma, mesothelioma; Gastrointestinal: esophagus [squamous cell carcinoma, adenocarcinoma, leiomyosarcoma, lymphoma], stomach [carcinoma, lymphoma, leiomyosarcoma], pancreas [ductal adenocarcinoma, insulinoma, glucagonoma, gastrinoma, carcinoid tumors, VIPoma], small bowel [adenocarcinoma, lymphoma, carcinoid tumors, Kaposi's sarcoma, leiomyoma, hemangioma, lipoma, neurofibroma, fibroma], large bowel [adenocarcinoma, tubular adenoma, villous adenoma, hamartoma, leiomyoma]; Genitourinary tract: kidney [adenocarcinoma, Wilms tumor (nephroblastoma), lymphoma, leukemia], bladder and urethra [squamous cell carcinoma, transitional cell carcinoma, adenocarcinoma], prostate [adenocarcinoma, sarcoma], testis [seminoma, teratoma, embryonal carcinoma, teratocarcinoma, choriocarcinoma, sarcoma, Leydig cell tumor, fibroma, fibroadenoma, adenomatoid tumors, lipoma]; Liver: hepatocellular carcinoma, cholangiocarcinoma, hepatoblastoma, angiosarcoma, hepatocellular adenoma, hemangioma; Bone: osteogenic sarcoma [osteosarcoma], fibrosarcoma, malignant fibrous histiocytoma, chondrosarcoma, Ewing's sarcoma, malignant lymphoma [reticulum cell sarcoma], multiple myeloma, malignant giant cell tumor, chordoma, osteochondroma [osteocartilaginous exostoses], benign chondroma, chondroblastoma, chondromyxoid fibroma, osteoid osteoma and giant cell tumors; Nervous system: skull [osteoma, hemangioma, granuloma, xanthoma, Paget's disease of bone], meninges [meningioma, meningiosarcoma, gliomatosis], brain [astrocytoma, medulloblastoma, glioma, ependymoma, germinoma (pinealoma), glioblastoma multiforme, oligodendroglioma, schwannoma, retinoblastoma, congenital tumors], spinal cord [neurofibroma, meningioma, glioma, sarcoma]; Gynecological: uterus [endometrial carcinoma], cervix [cervical carcinoma, pre-invasive cervical dysplasia], ovaries [ovarian carcinoma (serous cystadenocarcinoma, mucinous cystadenocarcinoma, endometrioid carcinoma, clear cell adenocarcinoma, unclassified carcinoma), granulosa-theca cell tumors, Sertoli-Leydig cell tumors, dysgerminoma, malignant teratoma and other germ cell tumors], vulva [squamous cell carcinoma, intraepithelial carcinoma, adenocarcinoma, fibrosarcoma, melanoma], vagina [clear cell carcinoma, squamous cell carcinoma, sarcoma botryoides (embryonal rhabdomyosarcoma), fallopian tubes [carcinoma]; Hematologic: blood [myeloid leukemia (acute and chronic), acute lymphoblastic leukemia, chronic lymphocytic leukemia, myeloproliferative diseases, multiple myeloma, myelodysplastic syndrome], Hodgkin's disease, non-Hodgkin's lymphoma (malignant lymphoma); Skin: malignant melanoma, basal cell carcinoma, squamous cell carcinoma, Kaposi's sarcoma, nevi, dysplastic nevi, lipoma, angioma, dermatofibroma, keloids, psoriasis; and Adrenal glands: neuroblastoma.

Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

In a specific embodiment, it may be desirable to administer the pharmaceutical compositions of the invention locally to the area in need of treatment; this may be achieved, for example, and not by way of limitation, by local infusion during surgery, by topical application, by injection, by means of a catheter, or by means of an implant, said implant being of a porous, non-porous, or gelatinous material, including membranes, such as sialastic membranes, or fibers or co-polymers such as Elvax (see Ruan et al, 1992, Proc Natl Acad Sci USA, 89:10872-10876). In one embodiment, administration can be by direct injection by aerosol inhaler.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J. (1983) Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, i.e., the airways, thus requiring only a fraction of the systemic dose (see, e.g., Goodson, in Medical Applications of Controlled Release (1984) supra, vol. 2, pp. 115-138). Other suitable controlled release systems are discussed in the review by Langer (1990) Science 249:1527-1533.

Targeting Cells in which Apoptosis is Desirable

As used herein, the compounds of the present invention may be used for delivery to cells in which apoptosis is desirable, for example, tumor cells, or for “targeting” of neoplasms. In this sense, the “targeting” of compounds refers to a drug conjugate which increases the ratio of the area under the curve (AUC) in neoplastic tissue to the area under the curve (AUC) in whole blood for the drug conjugate in comparison to the parent compound administered under the same conditions. Targeting of the compounds of the present invention to the cells in which apoptosis is desirable or to cells in which apoptosis is undesirable may also be accomplished by conjugating the compounds of the invention to an antibody that is specific for the MAC itself or for individual components comprising the MAC. Methods for performing such conjugations are known to those skilled in the art.

Formulations may also be targeted to a neoplasm, e.g., liposomal formulations, pegylated formulations, or microencapsulated formulations, resulting in an increase in the AUCneoplasm/AUCblood ratio for the formulation in comparison to the compounds administered as a non-particulate formulation. Neoplastic targeting, with concomitant long neoplasm exposure times, can increase the proportion of neoplasm that do not move into cell cycle division when drug concentrations are high. Desirably the AUCneoplasm/AUCblood ratio is increased by 5%, 10%, 20%, 30%, 40%, 50%, 60%, 70%, 80%, 90%, or even 95%.

Linkers

A linker component of the invention is, at its simplest, a bond between a compound of the invention and a group that is bulky or charged. The linker provides a linear, cyclic, or branched molecular skeleton having pendant groups covalently linking a compound of the invention to a group that is bulky or charged.

Thus, the linking of a compound of the invention to a group that is bulky or charged is achieved by covalent means, involving bond formation with one or more functional groups located on the compound of the invention and the bulky or charged group. Examples of chemically reactive functional groups which may be employed for this purpose include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

The covalent linking of a compound of the invention and a group that is bulky or charged may be effected using a linker which contains reactive moieties capable of reaction with such functional groups present in the compound of the invention and the bulky or charged group. For example, a hydroxyl group of the compound of the invention may react with a carboxyl group of the linker, or an activated derivative thereof, resulting in the formation of an ester linking the two.

Examples of moieties capable of reaction with sulfhydryl groups include α-haloacetyl compounds of the type XCH₂CO— (where X═Br, Cl or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but may additionally be useful in coupling to amino groups under certain conditions. Reagents such as 2-iminothiolane (Traut et al., Biochemistry 12:3266 (1973)), which introduce a thiol group through conversion of an amino group, may be considered as sulfhydryl reagents if linking occurs through the formation of disulphide bridges.

Examples of reactive moieties capable of reaction with amino groups include, for example, alkylating and acylating agents. Representative alkylating agents include:
(i) α-haloacetyl compounds, which show specificity towards amino groups in the absence of reactive thiol groups and are of the type XCH₂CO— (where X═Cl, Br or I), for example, as described by Wong Biochemistry 24:5337 (1979);
(ii) N-maleimide derivatives, which may react with amino groups either through a Michael type reaction or through acylation by addition to the ring carbonyl group, for example, as described by Smyth et al., J. Am. Chem. Soc. 82:4600 (1960) and Biochem. J. 91:589 (1964);
(iii) aryl halides such as reactive nitrohaloaromatic compounds;
(iv) alkyl halides, as described, for example, by McKenzie et al., J Protein Chem. 7:581 (1988);
(v) aldehydes and ketones capable of Schiff's base formation with amino groups, the adducts formed usually being stabilized through reduction to give a stable amine;
(vi) epoxide derivatives such as epichlorohydrin and bisoxiranes, which may react with amino, sulfhydryl, or phenolic hydroxyl groups;
(vii) chlorine-containing derivatives of s-triazines, which are very reactive towards nucleophiles such as amino, sufhydryl, and hydroxyl groups;
(viii) aziridines based on s-triazine compounds detailed above, e.g., as described by Ross, J. Adv. Cancer Res. 2:1 (1954), which react with nucleophiles such as amino groups by ring opening;
(ix) squaric acid diethyl esters as described by Tietze, Chem. Ber. 124:1215 (1991); and
(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include:
(i) isocyanates and isothiocyanates, particularly aromatic derivatives, which form stable urea and thiourea derivatives respectively;
(ii) sulfonyl chlorides, which have been described by Herzig et al., Biopolymers
(iii) acid halides;
(iv) active esters such as nitrophenylesters or N-hydroxysuccinimidyl esters;
(v) acid anhydrides such as mixed, symmetrical, or N-carboxyanhydrides;
(vi) other useful reagents for amide bond formation, for example, as described by M. Bodansky, Principles of Peptide Synthesis, Springer-Verlag, 1984;
(vii) acylazides, e.g. wherein the azide group is generated from a preformed hydrazide derivative using sodium nitrite, as described by Wetz et al., Anal. Biochem. 58:347 (1974); and
(viii) imidoesters, which form stable amidines on reaction with amino groups, for example, as described by Hunter and Ludwig, J. Am. Chem. Soc. 84:3491 (1962). Aldehydes and ketones may be reacted with amines to form Schiff's bases, which may advantageously be stabilized through reductive amination. Alkoxylamino moieties readily react with ketones and aldehydes to produce stable alkoxamines, for example, as described by Webb et al., in Bioconjugate Chem. 1:96 (1990).

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through O-acylurea formation followed by amide bond formation, may also be employed.

It will be appreciated that functional groups in the compound of the invention and/or the bulky or charged group may, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as .alpha.-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

So-called zero-length linkers, involving direct covalent joining of a reactive chemical group of the compound of the invention with a reactive chemical group of the bulky or charged group without introducing additional linking material may, if desired, be used in accordance with the invention. For example, a ring nitrogen of the compound of the invention can be linked directly via an amide bond to the charged or bulky group.

Most commonly, however, the linker will include two or more reactive moieties, as described above, connected by a spacer element. The presence of such a spacer permits bifunctional linkers to react with specific functional groups within the compound of the invention and the bulky or charged group, resulting in a covalent linkage between the two. The reactive moieties in a linker may be the same (homobifunctional linker) or different (heterobifunctional linker, or, where several dissimilar reactive moieties are present, heteromultifunctional linker), providing a diversity of potential reagents that may bring about covalent attachment between the compound of the invention and the bulky or charged group.

Bulky Groups

The function of a bulky group is to increase the size of the compound of the invention sufficiently to inhibit passage across the blood-brain barrier, if it is desirous to do so. Bulky groups capable of inhibiting passage of the compound of the invention across the blood-brain barrier include those having a molecular weight greater than 200, 300, 400, 500, 600, 700, 800, 900, or 1000-daltons. Desirably, these groups are attached through a ring nitrogen of the compound of the invention.

Desirably, a bulky group is selected which enhances the cellular or neoplasm uptake of the conjugate. For example, certain peptides enable active translocation across the plasma membrane into cells (e.g., the Tat(49-57) peptide). Exemplary peptides which promote cellular uptake are disclosed, for example, by Wender et al., Proc. Natl. Acad. Sci. USA 97(24): 13003-8 (2000) and Laurent et al., FEBS Lett 443(1):61-5 (1999), incorporated herein by reference.

The bulky group may also be charged. For example, bulky groups include, without limitation, charged polypeptides, such as poly-arginine (guanidinium side chain), poly-lysine (ammonium side chain), poly-aspartic acid (carboxylate side chain), poly-glutamic acid (carboxlyate side chain), or poly-histidine (imidazolium side chain).

A charged polysaccharide that may also be used to promote neoplasm uptake of the compound of the invention. One polysaccharide useful for neoplasm targeting is hyaluronic acid or a low molecular weight fragments thereof (e.g. where n is 6-12). Certain neoplasms, including many that are found in the lung, overexpress the CD44 cell-surface marker. CD44 is found at low levels on epithelial, hemopoietic, and neuronal cells and at elevated levels in various carcinoma, melanoma, lymphoma, breast, colorectal, and lung neoplasm cells. This cell surface receptor binds to hyaluronic acid. Hyaluronic acid is a major component of the extracellular matrix, and CD44 is implicated in the metabolism of solubilized hyaluronic acid. CD44 appears to regulate lymphocyte adhesion to cells of the high endothelial venules during lymphocyte migration, a process that has many similarities to the metastatic dissemination of solid neoplasms. It is also implicated in the regulation of the proliferation of cancer cells. Hyaluronic acid conjugates can gain access to the neoplasm cells subsequent to extravasating into the neoplasm from the circulation, resulting in an enhanced concentration of the conjugate within the neoplasm. See, for example, Eliaz et al., Cancer Research 61:2592 (2001) and references cited therein. 48

The bulky group can be an antiproliferative agent used in the combinations of the invention. Such conjugates are desirable where the two agents should have matching pharmacokinetic profiles to enhance efficacy and/or to simplify the dosing regimen.

Charged Groups

The function of a charged group is to alter the charge of the compound of the invention sufficiently to inhibit passage across the blood-brain barrier. Desirably, charged groups are attached through a ring nitrogen of the compound of the invention.

A charged group may be cationic or an anionic. Charged groups include 3, 4, 5, 6, 7, 8, 9, 10, or more negatively charged moieties and/or 3, 4, 5, 6, 7, 8, 9, 10, or more positively charged moieties. Charged moieties include, without limitation, carboxylate, phosphodiester, phosphoramidate, borate, phosphate, phosphonate, phosphonate ester, sulfonate, sulfate, thiolate, phenolate, ammonium, amidinium, guanidinium, quaternary ammonium, and imidazolium moieties.

Therapy for Neoplastic Diseases

The compounds of the invention which open the MAC, thus leading to apoptotic cell death, may be formulated in compositions useful for the treatment of neoplasms. Therapy may be performed alone or in conjunction with another therapy (e.g., surgery, radiation therapy, chemotherapy, immunotherapy, anti-angiogenesis therapy, or gene therapy).

The duration of the therapy depends on the type of disease or disorder being treated, the age and condition of the patient, the stage and type of the patient's disease, and how the patient responds to the treatment. Therapy may be given in on-and-off cycles that include rest periods so that the patient's body has a chance to recovery from any as yet unforeseen side-effects. Therapy may also be given for a continuous period.

Therapy for Neurodegenerative Diseases

Blood-Brain Barrier

The compounds of the present invention, which exhibit the ability to prevent opening of the mitochondrial apoptosis-induced channel (MAC), or promote closure of the mitochondrial apoptosis-induced channel (MAC), are contemplated for use in treating diseases or conditions wherein apoptotic cell death is undesirable, such as in neurodegenerative diseases, or injuries of the nervous system, such as spinal cord injury or traumatic brain injury, or for treatment of cardiovascular diseases.

Agents of the invention that exert their physiological effect in vivo in the brain may be more useful if they gain access to target cells in the brain. Non-limiting examples of brain cells are neurons, glial cells (astrocytes, oligodendrocytes, microglia), cerebrovascular cells (muscle cells, endothelial cells), and cells that comprise the meninges. The blood brain barrier (“BBB”) typically restricts access to brain cells by acting as a physical and functional blockade that separates the brain parenchyma from the systemic circulation (see, e.g., Pardridge, et al., J. Neurovirol. 5(6), 556-69 (1999); Rubin, et al., Rev. Neurosci. 22, 11-28 (1999)). Circulating molecules are normally able to gain access to brain cells via one of two processes: lipid-mediated transport through the BBB by free diffusion, or active (or catalyzed) transport.

The agents of the invention may be formulated to improve distribution in vivo, for example as powdered or liquid tablet or solution for oral administration or as a nasal spray, nose drops, a gel or ointment, through a tube or catheter, by syringe, by packtail, by pledget, or by submucosal infusion. For example, the blood-brain barrier (BBB) excludes many highly hydrophilic agents. To ensure that the more hydrophilic therapeutic agents of the invention cross the BBB, they may be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties” or “targeting groups” or “transporting vectors”), thus providing targeted drug delivery (see, e.g., V. V. Ranade J. Clin. Pharmacol. 29, 685 (1989)). Likewise, the agents may be linked to targeting groups that facilitate penetration of the blood brain barrier.

To facilitate transport of agents of the invention across the BBB, they may be coupled to a BBB transport vector (for review of BBB transport vectors and mechanisms, see, Bickel, et al., Adv. Drug Delivery Reviews 46, 247-79 (2001)). Exemplary transport vectors include cationized albumin or the OX26 monoclonal antibody to the transferrin receptor; these proteins undergo absorptive-mediated and receptor-mediated transcytosis through the BBB, respectively. Natural cell metabolites that may be used as targeting groups, include putrescine, spermidine, spermine, or DHA. Other exemplary targeting moieties include folate or biotin (see, e.g., U.S. Pat. No. 5,416,016); mannosides (Umezawa, et al., Biochem. Biophys. Res. Commun. 153, 1038 (1988)); antibodies (P. G. Bloeman, et al., FEBS Lett. 357, 140 (1995); M. Owais, et al., Antimicrob. Agents Chemother. 39, 180 (1995)); surfactant protein A receptor (Briscoe, et al., Am. J. Physiol. 1233, 134 (1995)); gp120 (Schreier, et al., J. Biol. Chem. 269, 9090 (1994)); see also, K. Keinanen and M. L. Laukkanen, FEBS Lett. 346, 123 (1994); J. J. Killion and I. J. Fidler, Immunomethods 4, 273 (1994).

Examples of other BBB transport vectors that target receptor-mediated transport systems into the brain include factors such as insulin, insulin-like growth factors (“IGF-I,” and “IGF-II”), angiotensin II, atrial and brain natriuretic peptide (“ANP,” and “BNP”), interleukin I (“IL-1”) and transferrin. Monoclonal antibodies to the receptors that bind these factors may also be used as BBB transport vectors. BBB transport vectors targeting mechanisms for absorptive-mediated transcytosis include cationic moieties such as cationized LDL, albumin or horseradish peroxidase coupled with polylysine, cationized albumin or cationized immunoglobulins. Small basic oligopeptides such as the dynorphin analogue E-2078 and the ACTH analogue ebiratide may also cross the brain via absorptive-mediated transcytosis and are potential transport vectors.

Other BBB Transport Vectors Target Systems for Transporting Compounds Into the Brain.

Examples of such BBB transport vectors include hexose moieties, e.g., glucose and monocarboxylic acids, e.g., lactic acid and neutral amino acids, e.g., phenylalanine and amines, e.g., choline and basic amino acids, e.g., arginine, nucleosides, e.g., adenosine and purine bases, e.g., adenine, and thyroid hormone, e.g., triiodothyridine. Antibodies to the extracellular domain of nutrient transporters may also be used as transport vectors. Other possible vectors include angiotensin II and ANP, which may be involved in regulating BBB permeability.

In some cases, the bond linking the therapeutic agent to the transport vector may be cleaved following transport into the brain in order to liberate the biologically active agent. Exemplary linkers include disulfide bonds, ester-based linkages, thioether linkages, amide bonds, acid-labile linkages, and Schiff base linkages. Avidin/biotin linkers, in which avidin is covalently coupled to the BBB drug transport vector, may also be used. Avidin itself may be a drug transport vector.

Transcytosis, including receptor-mediated transport of compositions across the blood brain barrier, may also be suitable for the agents of the invention. Transferrin receptor-mediated delivery is disclosed in U.S. Pat. Nos. 5,672,683; 5,383,988; 5,527,527; 5,977,307; and 6,015,555. Transferrin-mediated transport is also known. P. M. Friden, et al., Pharmacol. Exp. Ther. 278, 1491-98 (1996); H. J. Lee, J. Pharmacol. Exp. Ther. 292, 1048-52 (2000). EGF receptor-mediated delivery is disclosed in Y. Deguchi, et al., Bioconjug. Chem. 10, 32-37 (1999), and transcytosis is described in A. Cerletti, et al., J. Drug Target. 8, 435-46 (2000). Insulin fragments have also been used as carriers for delivery across the blood brain barrier. M. Fukuta, et al., Pharm. Res. 11. 1681-88 (1994). Delivery of agents via a conjugate of neutral avidin and cationized human albumin has also been described. Y. S. Kang, et al., Pharm. Res. 1, 1257-64 (1994).

Other modifications for enhancing penetration of the agents of the invention across the blood brain barrier may be accomplished using methods and derivatives known in the art. For example, U.S. Pat. No. 6,024,977 discloses covalent polar lipid conjugates for targeting to the brain and central nervous system. U.S. Pat. No. 5,017,566 discloses cyclodextrin derivatives comprising inclusion complexes of lipoidal forms of dihydropyridine redox targeting moieties. U.S. Pat. No. 5,023,252 discloses the use of pharmaceutical compositions comprising a neurologically active drug and a compound for facilitating transport of the drug across the blood-brain barrier including a macrocyclic ester, diester, amide, diamide, amidine, diamidine, thioester, dithioester, thioamide, ketone or lactone. U.S. Pat. No. 5,024,998 discloses parenteral solutions of aqueous-insoluble drugs with cyclodextrin derivatives. U.S. Pat. No. 5,039,794 discloses the use of a metastatic tumor-derived egress factor for facilitating the transport of compounds across the blood-brain barrier. U.S. Pat. No. 5,112,863 discloses the use of N-acyl amino acid derivatives as antipsychotic drugs for delivery across the blood-brain barrier. U.S. Pat. No. 5,124,146 discloses a method for delivery of therapeutic agents across the blood-brain barrier at sites of increase permeability associated with brain lesions. U.S. Pat. No. 5,153,179 discloses acylated glycerol and derivatives for use in a medicament for improved penetration of cell membranes. U.S. Pat. No. 5,177,064 discloses the use of lipoidal phosphonate derivatives of nucleoside antiviral agents for delivery across the blood-brain barrier. U.S. Pat. No. 5,254,342 discloses receptor-mediated transcytosis of the blood-brain barrier using the transferrin receptor in combination with pharmaceutical compounds that enhance or accelerate this process. U.S. Pat. No. 5,258,402 discloses treatment of epilepsy with imidate derivatives of anticonvulsive sulfamate. U.S. Pat. No. 5,270,312 discloses substituted piperazines as central nervous system agents. U.S. Pat. No. 5,284,876 discloses fatty acid conjugates of dopamine drugs. U.S. Pat. No. 5,389,623 discloses the use of lipid dihydropyridine derivatives of anti-inflammatory steroids or steroid sex hormones for delivery across the blood-brain barrier. U.S. Pat. No. 5,405,834 discloses prodrug derivatives of thyrotropin releasing hormone. U.S. Pat. No. 5,413,996 discloses acyloxyalkyl phosphonate conjugates of neurologically-active drugs for anionic sequestration of such drugs in brain tissue. U.S. Pat. No. 5,434,137 discloses methods for the selective opening of abnormal brain tissue capillaries using bradykinin infused into the carotid artery. U.S. Pat. No. 5,442,043 discloses a peptide conjugate between a peptide having a biological activity and incapable of crossing the blood-brain barrier and a peptide which exhibits no biological activity and is capable of passing the blood-brain barrier by receptor-mediated endocytosis. U.S. Pat. No. 5,466,683 discloses water soluble analogues of an anticonvulsant for the treatment of epilepsy. U.S. Pat. No. 5,525,727 discloses compositions for differential uptake and retention in brain tissue comprising a conjugate of a narcotic analgesic and agonists and antagonists thereof with a lipid form of dihydropyridine that forms a redox salt upon uptake across the blood-brain barrier that prevents partitioning back to the systemic circulation.

Still further examples of modifications that enhance penetration of the blood brain barrier are described in International (PCT) Application Publication Number WO 85/02342, which discloses a drug composition comprising a glycerolipid or derivative thereof. PCT Publication Number WO 089/11299 discloses a chemical conjugate of an antibody with an enzyme which is delivered specifically to a brain lesion site for activating a separately-administered neurologically-active prodrug. PCT Publication Number WO 91/04014 discloses methods for delivering therapeutic and diagnostic agents across the blood-brain barrier by encapsulating the drugs in liposomes targeted to brain tissue using transport-specific receptor ligands or antibodies. PCT Publication Number WO 91/04745 discloses transport across the blood-brain barrier using cell adhesion molecules and fragments thereof to increase the permeability of tight junctions in vascular endothelium. PCT Publication Number WO 91/14438 discloses the use of a modified, chimeric monoclonal antibody for facilitating transport of substances across the blood-brain barrier. PCT Publication Number WO 94/01131 discloses lipidized proteins, including antibodies. PCT Publication Number WO 94/03424 discloses the use of amino acid derivatives as drug conjugates for facilitating transport across the blood-brain barrier. PCT Publication Number WO 94/06450 discloses conjugates of neurologically-active drugs with a dihydropyridine-type redox targeting moiety and comprising an amino acid linkage and an aliphatic residue. PCT Publication Number WO 94/02178 discloses antibody-targeted liposomes for delivery across the blood-brain barrier. PCT Publication Number WO 95/07092 discloses the use of drug-growth factor conjugates for delivering drugs across the blood-brain barrier. PCT Publication Number WO 96/00537 discloses polymeric microspheres as injectable drug-delivery vehicles for delivering bioactive agents to sites within the central nervous system. PCT Publication Number WO 96/04001 discloses omega-3-fatty acid conjugates of neurologically-active drugs for brain tissue delivery. PCT WO 96/22303 discloses fatty acid and glycerolipid conjugates of neurologically-active drugs for brain tissue delivery.

Screening Methods

The present invention also provides methods of identifying modulators of the MAC, comprising contacting a test compound with the MAC and detecting the activity of the MAC. Preferably, the methods of identifying modulators or screening assays employ cells containing endogenous MAC or alternatively, transformed host cells that express the MAC. Typically, such assays will detect changes in the activity of the MAC due to the test compound, thus identifying modulators of the MAC. Modulators of the MAC are useful in modulating apoptosis. Blockers or inhibitors of the MAC will prevent apoptosis and thereby be useful in the treatment of conditions whereby apoptosis is undesirable, such as in stroke, Alzheimer's disease, myocardial infarct, traumatic brain injury and spinal cord injury. Activators or agents that promote opening of the MAC will be useful in the treatment of diseases or conditions whereby apoptosis is desirable, such as in cancer or hyperproliferative disorders or conditions.

The MAC can be used in a patch clamp or other type of assays, such as the assays disclosed herein, to identify small molecules, antibodies, peptides, proteins, or other types of compounds that inhibit, block, or otherwise interact with the MAC. Such modulators identified by the screening assays can then be used for treatment of diseases or conditions in mammals wherein it is either desirable to promote apoptosis or desirable to inhibit apoptosis.

For example, host cells expressing the MAC can be employed in a cytochrome c efflux assay such as that described in the art (Cheng, E. H., Sheiko, T. V., Fisher, J. K., Craigen, W. J. and Korsmeyer, S. J. (2003), VDAC2 inhibits BAK activation and mitochondrial apoptosis. Science 301, 513-7). However, this assay can be modified by using an ELISA in lieu of a western blot. Additionally, the host cells expressing the MAC can be used in electrophysiological assays using patch clamp techniques as described (Guo, L., Pietkiewicz, D., Pavlov, E. V., Kasianowicz, J. J., Korsmeyer, S. J., Antonsson, B. and Kinnally, K. W. (2004) American Journal of Physiology, Cell Biology. 286, C1109-17; Pavlov, E. V., Priault, M., Pietkiewicz, D., Cheng, E.H.-Y., Antonsson, B., Manon, S., Korsmeyer, S. J., Mannella, C. A. and Kinnally, K. W. (2001) J. Cell Biology 155, 725-732; Lohret, T. A., Jensen, R. and Kinnally, K. W. (1997) J. Cell Biol. 137, 377-386). In general, a test compound is added to the assay and its effect on cytochrome c flux is determined or the test compound's ability to competitively bind to the channel is assessed. Test compounds having the desired effect on the channel are then selected. Modulators so selected can then be used for treating the conditions described herein. To examine the extent of inhibition, samples or assays comprising the MAC are treated with a potential activator or inhibitor compound and are compared to control samples without the test compound. Control samples (untreated with test compounds) arc assigned a relative MAC modulating activity value of 100%. Inhibition of channels comprising MAC is achieved when the MAC modulating activity value relative to the control is about 90%, preferably 50%, more preferably 25-0%. Activation of channels comprising MAC is achieved when the MAC modulating activity value relative to the control is 110%, more preferably 150%, most preferably at least 200-500% higher or 1000% or higher.

Effective Doses

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects can be used, care should be taken to design a delivery system that targets such compounds to the site of affected tissue in order to minimize potential damage to unaffected cells and, thereby, reduce side effects.

The data obtained from cell culture assays and animal studies can be used in formulating a dose range for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to optimize efficacious doses for administration to humans. Plasma levels can be measured by any technique known in the art, for example, by high performance liquid chromatography.

Thus, the amount of the compound of the invention which will be effective in the treatment of cancer or hyperproliferative disorders, or alternatively, stroke, Alzheimer's disease, myocardial infarction, traumatic brain injury and spinal cord injury can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. Normal dose ranges used for particular therapeutic agents and standard cancer treatments employed for specific diseases can be found in the Physicians' Desk Reference, 54^{th} Edition (2000) or in Cancer: Principles & Practice of Oncology, DeVita, Jr., Hellman, and Rosenberg (eds.) 2nd edition, Philadelphia, Pa.: J.B. Lippincott Co., 1985. By way of non-limiting example, suitable dose ranges for intravenous administration of propranolol, for example, are generally about 1 mg to about 3 mg administered under careful monitoring. Similarly, suitable oral doses for propranolol range from about 10 mg to about 30 mg three or four times daily to about 80 mg to 320 mg twice a day, three times a day or four times a day, depending on the indication. Also by way of non-limiting example, trifluoperazine, for example, may be effective at doses ranging from about 1 to 10 mg per day given orally. It is to be noted that dosages should be adjusted to the needs of the individual, and where possible, the lowest effective dosage should be used. Effective doses may be extrapolated from dose-response curves derived from in vitro or animal model test systems.

Assays for Modulators of MAC

The activity of a MAC modulator can be assessed using a variety of in vitro and in vivo assays. Preferably, the in vitro assays disclosed herein in the example section are used to identify MAC modulators for treatment of stroke, Alzheimer's disease, myocardial infarct, traumatic brain injury, spinal cord injury, cancer and hyperproliferative disorders. Such assays are used to test for inhibitors and activators of MAC. For example, it is desirable to identify compounds that reduce the uncontrolled growth of tumor cells in subjects by enhancing the opening of the mitochondrial apoptosis induced channel to allow for release of cytochrome c and other small molecules that act as a death signal by traversing gap junctions. Conversely, it is desirable to identify compounds that promote closure of the channel or prevent its opening in order to prevent neighboring cells from dying due to release of cytochrome c and other small molecule death signals in diseases or conditions whereby apoptosis is undesirable. Assays for modulatory compounds include, e.g., measuring current; measuring membrane potential; measuring flux of cytochrome c; measuring second messengers and transcription levels, measuring responses in appropriate animal models for tumor cell growth or prevention thereof, measuring responses in animal models of stroke, traumatic brain injury or spinal cord injury to look for protection of neurons from apoptotic cell death after either occlusion of the cerebral artery or after a contusion to the spinal cord; measuring ligand binding; and using, e.g., voltage-sensitive dyes, radioactive tracers, and patch-clamp electrophysiology.

Modulators of MAC are tested using biologically active MAC, either recombinant or naturally occurring. MAC can be isolated in vitro, co-expressed or expressed in a cell, or expressed in a membrane derived from a cell. It can be isolated from mitochondrial outer membrane preparations and fused with liposomes to create proteoliposomes using the method of Criado and Keller (Guo, L., Pietkiewicz, D., Pavlov, E. V., Kasianowicz, J. J., Korsmeyer, S. J., Antonsson, B. and Kinnally, K. W. (2004) American Journal of Physiology, Cell Biology. 286, C1109-17; Pavlov, E. V., Priault, M., Pietkiewicz, D., Cheng, E. H.-Y., Antonsson, B., Manon, S., Korsmeyer, S. J., Mannella, C. A. and Kinnally, K. W. (2001) J. Cell Biology 155, 725-732; Criado, M. and Keller, B. U. (1987) FEBS Letts 224, 172-176). In such assays, in order to test for an inhibitor of MAC, cytochrome c may be loaded into these proteoliposomes and the amount retained measured to be more than that of liposomes containing MAC without the inhibitor. One would then add test compounds to determine whether they block MAC (ie. whether they close the channel), the readout being the prevention of efflux of cytochrome c from the proteoliposomes. Such blocking would then be an indicator that the test compound would inhibit apoptosis. Such test compounds, once identified as being inhibitors of MAC, would then be tested in appropriate animal models of stroke or spinal cord injury or traumatic brain injury to determine their effectiveness in vivo as an apoptosis inhibitor. Conversely, if the test compound activates MAC (ie. opens the channel), as shown by enhancement of cytochrome c efflux from the proteoliposome, the test compound would be a potential enhancer of apoptosis. In one particular embodiment, t-Bid may induce the efflux of cytochrome c from untreated isolated mitochondria. Openers would then compare with the ability of the BH3-peptide tested previously for cyt c efflux. (Polster, B. M., Kinnally, K. W. and Fiskum, G. (2001).BH3 death domain peptide induces cell type-selective mitochondrial outer membrane permeability. J Biol Chem 276, 37887-94). Such test compound would then be assessed for anti-tumor activity in appropriate animal models. Samples or assays that are treated with a potential MAC inhibitor or activator are compared to control samples without the test compound, to examine the extent of modulation. Control samples (untreated with activators or inhibitors) are assigned a relative MAC modulation activity value of 100. Inhibition of MAC is achieved when the MAC modulation activity value relative to the control is about 90%, preferably 50%, more preferably 25-0%. Activation of MAC is achieved when the MAC modulation activity value relative to the control is 110%, more preferably 150%, more preferably 200-500% higher, preferably 1000% or higher.

Assays for compounds capable of inhibiting or increasing ion flux through the channel proteins can be performed by application of the compounds to a bath solution in contact with and comprising mitochondria having a channel of the present invention (see, e.g., Blatz et al., Nature 323:718-720 (1986); Park, J. Physiol. 481:555-570 (1994)). Generally, the compounds to be tested are present in the range from 1 pM to 100 mM. Cells expressing the channel can express recombinant MAC (e.g., CHO cells or Xenopus cells) or endogenous MAC in their mitochondria.

The effects of a test compound can be measured by a toxin binding assay. When the functional consequences are determined using intact cells or animals, one can also measure a variety of effects such as transmitter release (e.g., dopamine), hormone release (e.g., insulin), transcriptional changes to both known and uncharacterized genetic markers (e.g., northern blots), cell volume changes (e.g., in red blood cells), immunoresponses (e.g., T cell activation), changes in cell metabolism such as cell growth or pH changes, and changes in intracellular second messengers such as Ca²⁺, or cyclic nucleotides. Preferably human MAC are used in the assays of the invention. Optionally, MAC orthologs from other species such as rat or mouse, preferably a mammalian species, are used in the assays of the invention.

Modulators

The chemical compounds of the invention, which modulate MAC, are made according to methodology well known to those of skill in the art.

The compounds tested as modulators of MAC can be any small chemical compound, or a biological entity, such as a protein, an antibody, a sugar, nucleic acid or lipid. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepincs, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N. J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

In one embodiment, the invention provides solid phase based in vitro assays in a high throughput format, where the cell or tissue expressing a MAC is attached to a solid phase substrate. In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100 to about 1500 different compounds. It is possible to assay several different plates per day; assay screens for up to about 6,000-20,000 different compounds is possible using the integrated systems of the invention.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Modulation of MAC with Various Compounds

Materials and Methods

Cells and Growth Conditions

Hematopoietic FL5.12 cells are a mouse leukemia cell line that enters apoptosis after withdrawal of the growth factor interleukin-3 (IL-3) (Gross, A., Jockel, J., Wei, M. C. and Korsmeyer, S. J. (1998). Parental FL5.12 cells were cultured as previously described (Gross, A., Jockel, J., Wei, M. C. and Korsmeyer, S. J. (1998) EMBO J. 17, 3878-85) in Iscove's Modified Eagle Media (IMEM), 10% Fetal Bovine Serum, 10% WEHI-3B supplement (filtered supernatant of WEHI-3B cells secreting IL-3). Cultures were kept below 1.5 million cells/ml. Cells were washed three times in media without IL-3 (WEHI supplement) to induce apoptosis twelve hours prior to the isolation of mitochondria (Zamzami, N., Susin, S. A., Marchetti, P., Hirsch, T., Gomez-Monterrey, I., Castedo, M. and Kroemer, G. (1996). Mitochondrial control of nuclear apoptosis. Journal of Experimental Medicine. 183, 1533-44; Mootha, V., Wei, M., Buttle, K., Scorrano, L., Panoutsakopoulou, V., Mannella, C. and Korsmeyer, S. (2001). A reversible component of mitochondrial respiratory dysfunction in apoptosis can be rescued by exogenous cytochrome c. EMBO Journal 20, 661-671). Bax translocates and oligomerizes in mitochondria within twelve hours after IL-3 withdrawal (Gross, A., Jockel, J., Wei, M. C. and Korsmeyer, S. J. (1998). Enforced dimerization of BAX results in its translocation, mitochondrial dysfunction and apoptosis. EMBO J. 17, 3878-85; Gross, A., McDonnell, J. and Korsmeyer, S. (1999).Bcl-2 family members and the mitochondria in apoptosis. Genes & Devel. 13, 1899-1911. These events immediately precede release of pro-apoptotic factors, e.g., cytochrome c (Gross, A., Jockel, J., Wei, M. C. and Korsmeyer, S. J. (1998).Enforced dimerization of BAX results in its translocation, mitochondrial dysfunction and apoptosis. EMBO J. 17, 3878-85), which in turn, is prevented by anti-apoptotic Bcl-2 (Gross, A., Pilcher K., Blachly-Dyson, E., Basso, E., Jockel, J., Bassik, M. C., Korsmeyer, S. J. and Forte, M. (2000). Biochemical and genetic analysis of the mitochondrial response of yeast to BAX and BCL-X(L). Mol. Cell Biol. 20, 3125-26; Goping, I. S., Gross, A., Lavoie, J. N., Nguyen, M., Jemmerson, R., Roth, K., Korsmeyer, S. J. and Shore, G. C. (1998). Regulated targeting of BAX to mitochondria. J. Cell Biology 143, 207-215). Membrane blebbing (video) and loss of plasma membrane integrity (Propidium Iodide staining) occur at 36-48 hours after IL-3 withdrawal. Annexin-V staining begins at ˜36 hours.

Isolation of Mitochondria and Preparation of Proteoliposomes

Mitochondria were isolated from 2-15 g of FL5.12 cells as previously described for outer membrane preparations (Guo, L., Pietkiewicz, D., Pavlov, E. V., Kasianowicz, J. J., Korsmeyer, S. J., Antonsson, B. and Kinnally, K. W. (2004) American Journal of Physiology, Cell Biology. 286, C1109-17; Pavlov, E. V., Priault, M., Pietkiewicz, D., Cheng, E.H.-Y., Antonsson, B., Manon, S., Korsmeyer, S. J., Mannella, C. A. and Kinnally, K. W. (2001) J. Cell Biology 155, 725-732). Outer membranes were stripped from the inner membranes by French pressing isolated mitochondria using modifications of the method of Decker and Greenawalt (Decker, G. L. and Greenawalt, J. W. (1977) J. Ultrastr. Res. 59, 44-56) as described in (Pavlov, E. V., Priault, M., Pietkiewicz, D., Cheng, E.H.-Y., Antonsson, B., Manon, S., Korsmeyer, S. J., Mannella, C. A. and Kinnally, K. W. (2001) J. Cell Biology 155, 725-732). French pressing was done at 2000 PSI in 460 mM Mannitol 140 mM Sucrose 2 mM EDTA 10 mM HEPES pH 7.4. The pressed suspension was diluted 1:1 with 230 mM Mannitol 70 mM sucrose 1 mM EDTA 5 mM HEPES pH 7.4 and centrifuged at 10,000 rpm (12,000 g) for 10 minutes. Outer membranes were separated from inner membranes as described by Mannella (Mannella, C. A. (1982) J. Cell Biol. 94, 680-687).

Proteoliposomes were formed by a modification of the method of Criado and Keller (Guo, L., Pietkiewicz, D., Pavlov, E. V., Kasianowicz, J. J., Korsmeyer, S. J., Antonsson, B. and Kinnally, K. W. (2004) American Journal of Physiology, Cell Biology. 286, C1109-17; Pavlov, E. V., Priault, M., Pietkiewicz, D., Cheng, E.H.-Y., Antonsson, B., Manon, S., Korsmeyer, S. J., Mannella, C. A. and Kinnally, K. W. (2001) J. Cell Biology 155, 725-732; Criado, M. and Keller, B. U. (1987) FEBS Letts 224, 172-176). Briefly, small liposomes were formed by sonication of lipid (Sigma Type IV-S soybean L-α-phosphatidylcholine) in water. Mitochondrial outer membranes (5-10 μg protein) and small liposomes (˜1 mg lipid) were mixed with 5 mM HEPES pH 7.4 and dotted on a glass slide. Samples were dehydrated ˜3 hours and re-hydrated overnight with 150 mM KCl 5 mM HEPES pH 7.4 at 4° C. Proteoliposomes were harvested with ˜0.5 ml of the same media and stored at −80° C.

Immunoblotting

Proteins were separated by SDS PAGE and electro-transferred onto PVDF membranes. Indirect immuno-detection employed chemiluminescence (Amersham) using HRP-coupled secondary antibodies. Mitochondrial outer and inner membranes (0.5-2 μg per lane) were probed with primary antibodies against mammalian VDAC1 (Calbiochem 31-HL, 1:2500), cytochrome oxidase subunit IV (Molec. Probes A-643361, 1:1000) and a secondary anti-rabbit or anti-mouse antibody (Jackson Immunoresearch, 1:5000).

Patch Clamp Analysis

Patch-clamp procedures and analysis used were previously described (Guo, L. et al. (2004), American J. Physiology, Cell Biology, 286, C1109-17; Pavlov, E. V. et al. (2001), J. Cell Biology, 155: 725-732; Lohret, T. A. et al. (1997), J. Cell Biology, 137: 377-386). Briefly, membrane patches were excised from proteoliposomes containing purified mitochondrial outer membranes after formation of a giga-seal using micropipettes with ˜0.4 μm diameter tips and resistances of 10-20 MΩ at room temperature. Unless otherwise stated, the solution was symmetrical 150 mM KCl, 5 mM HEPES pH 7.4. Voltage clamp was performed with the excised configuration of the patch-clamp technique (Hamill, O. P., Marty, A., Neher, E., Sakmann, B. and Sigworth, F. J. (1981) Pflüigers Archives-European Journal of Physiology 381, 85-100) using an Axopatch 200 amplifier. Voltages are reported as pipette potentials. The conductance was typically determined from total amplitude histograms of 30 seconds of current traces at +20 mV. MAC activity is distinct from VDAC and TOM channels (Pavlov, E. V., Priault, M., Pietkiewicz, D., Cheng, E.H.-Y., Antonsson, B., Manon, S., Korsmeyer, S. J., Mannella, C. A. and Kinnally, K. W. (2001) J. Cell Biology 155, 725-732). Currents were assigned as MAC if the conductance was >1.5 nS and voltage independent. pClamp version 8 (Axon Instru.) and WinEDR v2.3.3 (Strathclyde Electrophysiological Software; courtesy of J. Dempster, U. of Strathclyde, UK) were used for current analysis. Hill coefficients were determined as described by Moczydlowski [20].

Results

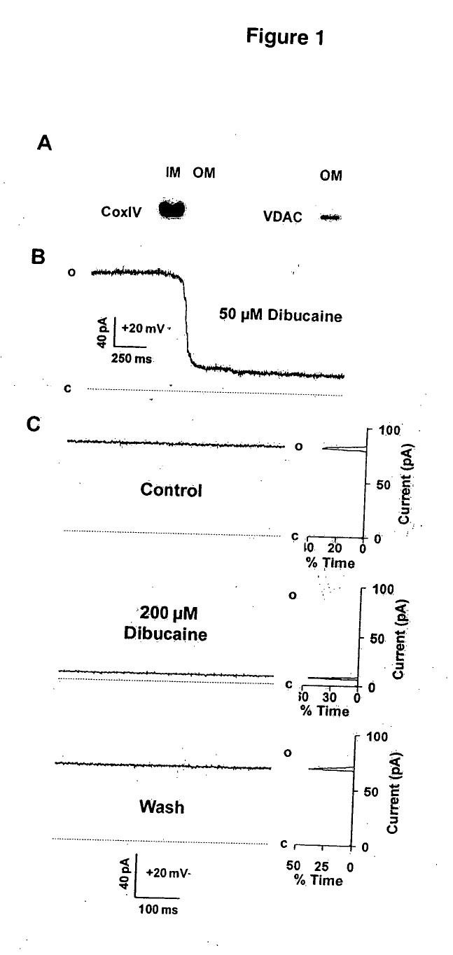

MAC is a potential therapeutic target because of its putative role in the commitment step of apoptosis, i.e., the release of cytochrome c. However, there is no pharmacological profile for MAC at this time. Apoptotic hematopoietic cells were used in this study to investigate the effect of a variety of agents on MAC activity. Apoptosis was induced by withdrawal of interleukin-3 (IL-3) from the leukemia cell line FL5.12. MAC activity is detected and cytochrome c is released from the mitochondria of these cells twelve hours after IL-3 withdrawal (Guo, L., Pietkiewicz, D., Pavlov, E. V., Kasianowicz, J. J., Korsmeyer, S. J., Antonsson, B. and Kinnally, K. W. (2004) American Journal of Physiology, Cell Biology. 286, C1109-17; Pavlov, E. V., Priault, M., Pietkiewicz, D., Cheng, E.H.-Y., Antonsson, B., Manon, S., Korsmeyer, S. J., Mannella, C. A. and Kinnally, K. W. (2001) J. Cell Biology 155, 725-732; Gross, A., Jockel, J., Wei, M. C. and Korsmeyer, S. J. (1998) EMBO J. 17, 3878-85). Outer membranes were purified from mitochondria isolated at this time. The western blot of FIG. 1A shows the presence of the outer membrane protein VDAC and essentially none of the inner membrane protein cytochrome oxidase subunit IV in the preparations; there is little contamination of the outer membranes by inner membranes. MAC activity was examined using patch clamp techniques on proteoliposomes formed by the fusion of these outer membranes with liposomes (See Methods). MAC is a heterogeneous channel with a variable high conductance and several substates. In this study, the effects of a variety of pharmacological agents were determined on MAC activity with a conductance of 1.5-5 nS and a long-lived open state (Guo, L., Pietkiewicz, D., Pavlov, E. V., Kasianowicz, J. J., Korsmeyer, S. J., Antonsson, B. and Kinnally, K. W. (2004) American Journal of Physiology, Cell Biology. 286, C1109-17; Pavlov, E. V., Priault, M., Pietkiewicz, D., Cheng, E.H.-Y., Antonsson, B., Manon, S., Korsmeyer, S. J., Mannella, C. A. and Kinnally, K. W. (2001) J. Cell Biology 155, 725-732).

MAC is exquisitely regulated by Bcl-2 family proteins but the molecular identity of this channel is not yet known. MAC is never detected in apoptotic cells overexpressing Bcl-2. Interestingly, MAC-like activity is detected in yeast expressing Bax and in membrane patches containing recombinant Bax (Pavlov, E. V., Priault, M., Pietkiewicz, D., Cheng, E.H.-Y., Antonsson, B., Manon, S., Korsmeyer, S. J., Mannella, C. A. and Kinnally, K. W. (2001) J. Cell Biology 155, 725-732). These findings suggest Bax might be involved with MAC activity. Dibucaine and propranolol (100-200 μM) prevent the release of cytochrome c induced by recombinant Bax plus t-Bid or BH3 peptide (Polster, B. M., Basanez, G., Young, M., Suzuki, M. and Fiskum, G. (2003) J Neurosci 23, 2735-43), but they do not prevent Bax insertion into membranes.

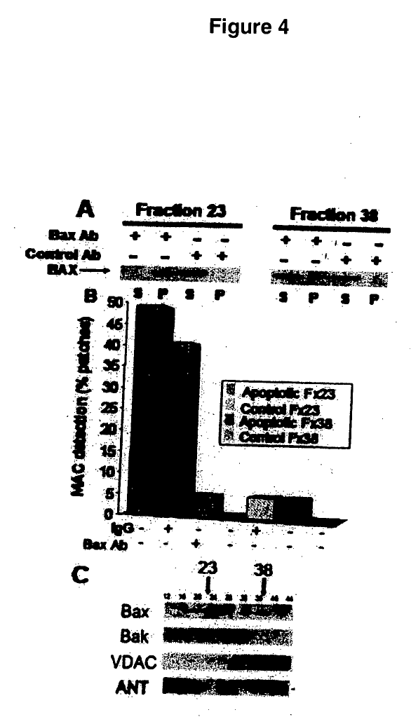

Current data establishes that Bax is integral to at least some of the MAC activity as immunoprecipitation with anti-Bax antibodies depletes MAC activity from a partially purified fraction of this preparation from apoptotic HeLa cells. Bax antibodies clear fractions of Bax from the supernatants of fraction 23 (FIG. 4A) from HeLa cells treated with staurosporine. Bax is present as a high molecular weight oligomer/complex in the mitochondrial membrane of apoptotic cells. (Antonsson, B., Montessuit, S., Sanchez, B. and Martinou, J. C. (2001), J. Biological Chemistry 276, 11615-11623). This fraction contains oligomeric Bax and Bak as well as MAC activity, but has no detectable VDAC or adenine nucleotide translocator (FIG. 4C). Immunoprecipitation of fraction 23 with a control IgG did not significantly reduce the detection of MAC. In contrast, immunoprecipitation with anti-Bax antibodies significantly reduced the activity as MAC was detected in only 1 of 21 patches (FIG. 4). The concomitant loss of MAC activity and Bax protein by immunoprecipitation with anti-Bax antibodies indicates that Bax is integral to at least some of the MAC activity.

Fraction 38 has little MAC activity but higher levels of Bax than fraction 23. Fraction 38 from control and apoptotic cells have 0.8 and 0.85 ng Bax/μg protein and <5% of patches have MAC while Fraction 23 from control and apoptotic cells have 0 (undetectable) and 0.3 ng Bax/μg protein, respectively (data not shown). MAC was not detected in the control fraction 23 but ˜half the patches had MAC activity in the apoptotic fraction 23. Taken together, these findings indicate MAC activity is associated with Bax oligomers, not monomers.

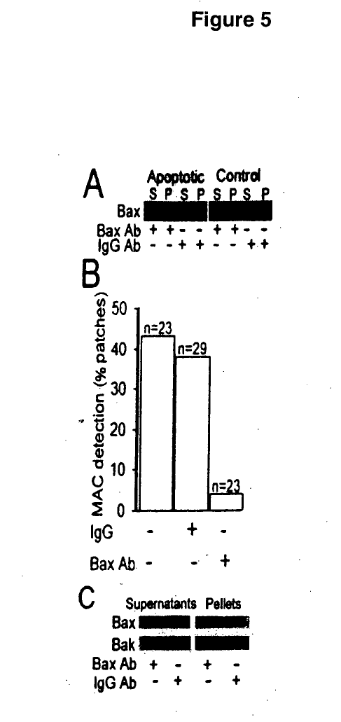

Current data also shows that Bax antibodies deplete whole mitochondrial lysates of most of the MAC activity (FIG. 5), which is consistent with the hypothesis that Bax is a component of MAC. These Bax antibodies recognize activated Bax (Mikhailov, V., Mikhailov, M., Pulkrabek, D. J., Dong, Z., Venkatachalam, M. A. and Saikumar, P. (2001), Bcl-2 prevents bax oligomerization in the mitochondrial outer membrane. J. Biol. Chem. 276, 18361-18374; Hsu, Y. T. and Youle, R. J. (1997), Nonionic detergents induce dimerization among members of the Bcl-2 family. J Biol Chem 272, 13829-34) (FIG. 4) and pull down only about 20% of the total Bax from apoptotic lysates. These experiments establish that Bax is component of most MAC, as the functional state of MAC has been attained in these samples.

The effect of dibucaine on MAC was examined because Bax may be a component of the MAC. As shown in FIG. 1B, the current flow through MAC is rapidly reduced upon perfusion of the bath with 50 μM dibucaine. The blockade of MAC by dibucaine is reversible as removal of dibucaine from the bath usually restores the high conductance. The total amplitude histograms and current traces of FIG. 1C illustrate this reversible inhibition in another patch containing MAC. The blockade is not voltage dependent as the current voltage curves are linear between +40 mV and −40 mV (not shown). The blockade of MAC by dibucaine is similar in partially purified fractions of MAC from apoptotic HeLa cells treated with staurosporine (unpublished results of Dejean, Martinez-Caballero, Antonsson & Kinnally).

Dibucaine causes a fast blockade of MAC, similar to the effects of diethylamine on sodium channels (Zamponi, G. W. and French, R. J. (1993) Biophys J 65, 2335-47). These blockade events are rapid as there is no discernable increase in noise at 2 kHz accompanying the decrease in conductance in the presence of dibucaine (not shown).

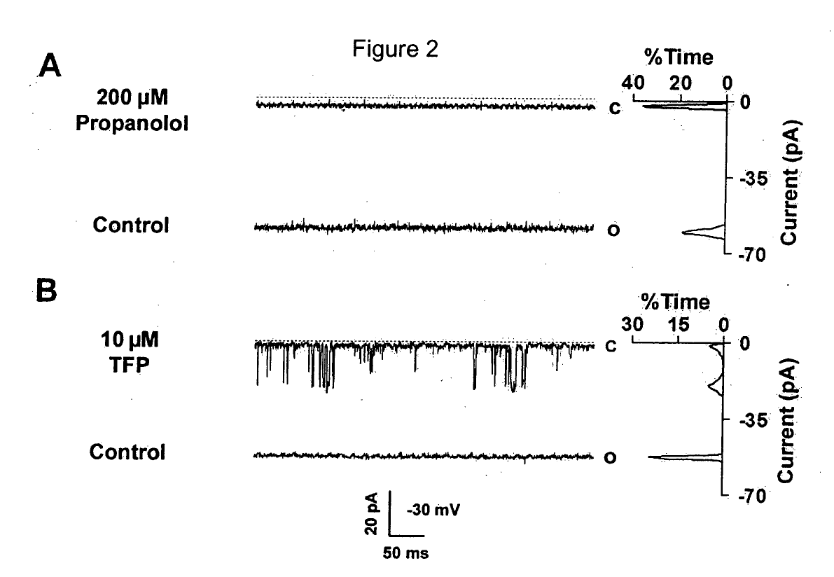

Like dibucaine, propranolol blocks the release of cytochrome c induced by Bax plus either a synthetic BH3 peptide or t-Bid (Polster, B. M., Basanez, G., Young, M., Suzuki, M. and Fiskum, G. (2003) J Neurosci 23, 2735-43). Propranolol also blocks the current flow through MAC as shown in the current traces and amplitude histograms of FIG. 2A. The inhibition of MAC conductance by propranolol is not voltage dependent as the current voltage curves are linear between +40 mV and −40 mV (not shown). In contrast to dibucaine, the effects of propranolol on MAC activity are not reversible as removal by perfusion of the bath does not restore MAC's conductance. Trifluoperazine also blocks MAC as shown in the current traces and amplitude histograms of FIG. 2B. Like propranolol, the blockade by trifluoperazine is not voltage dependent or reversible.

The mechanism of blockade of MAC by dibucaine is not the same as that of trifluoperazine and propranolol. Propranolol and trifluoperazine decrease the conductance of MAC, but the effects are not reversible. Repeated washing out of trifluoperazine and propranolol does not result in a re-opening of the channel. Therefore, the mechanism(s) underlying the effects of trifluoperazine and propranolol are likely either a tight binding that “plugs” the pore of MAC or a destabilization of the open state. The latter is more likely as the effects are not reversed several minutes after removal of the agents. Hence, the effects of trifluoperazine and propranolol are similar to the Type 2 effects of cytochrome c (Guo, L., Pietkiewicz, D., Pavlov, E. V., Kasianowicz, J. J., Korsmeyer, S. J., Antonsson, B. and Kinnally, K. W. (2004) American Journal of Physiology, Cell Biology. 286, C1109-17).

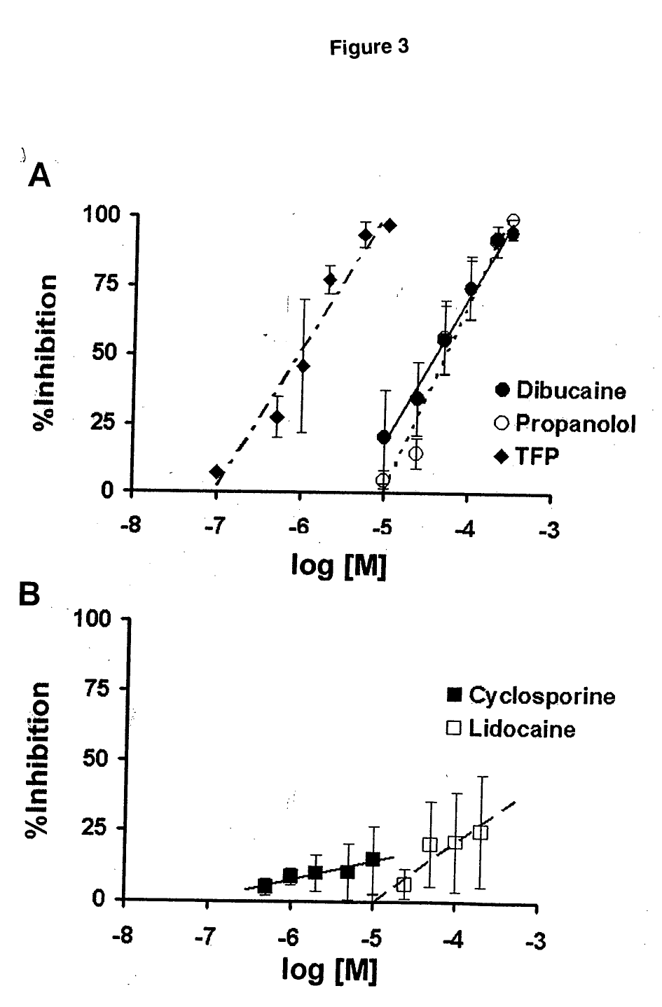

The three pharmacological agents inhibit MAC in a dose-dependent manner as shown in % inhibition curves of FIG. 3A. The IC₅₀ for trifluoperazine, propranolol, and dibucaine blockade of MAC (and Hill coefficients) are 0.9 μM (1.4±0.2), 52 μM (2.1±0.2), and 39 μM (1.3±0.1). While the mechanism of blockade for the three agents is not identical, the Hill coefficients for all of these inhibitors are more than 1. Typically, this finding indicates there is some degree of cooperativity involved in the blockade for each of these cationic amphiphilic drugs. Cyclosporine A and lidocaine have limited effects on MAC activity as shown in FIG. 3B. The IC₅₀ for these agents are mM to M, which is much larger than that of dibucaine, propranolol and trifluoperazine. 300 μM lidocaine has little effect on MAC activity. Cyclosporine A (0.1-1 μM) blocks the permeability transition pore (PTP) in mitochondria (Lenartowicz, E., Bernardi, P. and Azzone, G. F. (1991) J Bioenerg Biomembr 23, 679-88; Broekemeier, K. M., Carpenter-Deyo, L., Reed, D. J. and Pfeiffer, D. R. (1992) FEBS Lett 304, 192-4; Szabo, I., Bernardi, P. and Zoratti, M. (1992) J Biol Chem 267, 2940-6). However, the putative PTP inhibitors trifluoperazine (10-20 μM) and dibucaine (50-100 μM) (Hoyt, K. R., Sharma, T. A. and Reynolds, I. J. (1997) Br J Pharmacol 122, 803-8) also block MAC, but the IC₅₀ for MAC is lower than it is for the PTP. Similarly, propranolol blocks MCC, an electrophysiological manifestation of the PTP, but the IC₅₀ is ˜700 μM (Antonenko, Y. N., Kinnally, K. W., Perini, S. and Tedeschi, H. (1991) FEBS Letters 285, 89-93) compared to ˜50 μM for MAC. Interestingly, trifluoperazine and propranolol block apoptosis in some cell lines (Freedman, A. M., Kramer, J. H., Mak, I. T., Cassidy, M. M. and Weglicki, W. B. (1991) Free Radic Biol Med 11, 197-206; Nieminen, A. L., Saylor, A. K., Tesfai, S. A., Herman, B. and Lemasters, J. J. (1995) Biochem J 307 (Pt 1), 99-106). Furthermore, trifluoperazine (10-20 μM) and dibucaine (50-100 μM) also block mitochondrial depolarization induced by glutamate in neurons (Hoyt, K. R., Sharma, T. A. and Reynolds, I. J. (1997) Br J Pharmacol.122, 803-8). Hoyt et al. (1997) interpreted this protection as an inhibition of the permeability transition by these agents. Nevertheless, 0.1-1 μM Cyclosporine A blocks the permeability transition pore (PTP) in mitochondria (Lenartowicz, E., Bernardi, P. and Azzone, G. F. (1991) J Bioenerg Biomembr 23, 679-88; Broekemeier, K. M., Carpenter-Deyo, L., Reed, D. J. and Pfeiffer, D. R. (1992) FEBS Lett 304, 192-4; Szabo, I., Bernardi, P. and Zoratti, M. (1992) J Biol Chem 267, 2940-6) and up to 10 μM has no effect on MAC activity. These findings suggest that MAC and the PTP are independent. While the IC₅₀ for trifluoperazine is in the high nanomolar range, none of these drugs are highly specific. In fact, trifluoperazine, propranolol and dibucaine also inhibit mitochondrial protein import (Pavlov, P. F. and Glaser, E. (1998) Biochem Biophys Res Commun 252, 84-91) and inhibit the permeability transition induced by signal peptides, which likely corresponds to opening of the protein import channel Tim (Polster, B. M., Basanez, G., Young, M., Suzuki, M. and Fiskum, G. (2003) J Neurosci 23, 2735-43; Kushnareva, Y. E., Polster, B. M., Sokolove, P. M., Kinnally, K. W. and Fiskum, G. (2001) Archives of Biochemistry & Biophysics 386, 251-60; Sokolove, P. M. and Kinnally, K. W. (1996) Archives of Biochemistry & Biophysics 336, 69-76). While lidocaine and dibucaine increase membrane fluidity (Jutila, A., Rytomaa, M. and Kinnunen, P. K. (1998) Mol Pharmacol 54, 722-32; Kingston, C., Ladha, S., Manning, R. and Bowler, K. (1993) Anticancer Res. 13, 2335-40), lidocaine has no effect on MAC. In contrast, there are conflicting reports of the effects of propranolol on membrane fluidity (Jutila, A., Rytomaa, M. and Kinnunen, P. K. (1998) Mol Pharmacol 54, 722-32; Varga, E., Szollosi, J., Antal, K., Kovacs, P. and Szabo, J. Z. (1999) Pharmazie 54, 380-4). As dibucaine and propranolol may have opposing effects on bilayer fluidity, MAC opening may be somehow sensitive to this parameter. Others have suggested a role for lipids in the release of cytochrome c (Siskind, L. J., Davoody, A., Lewin, N., Marshall, S. and Colombini, M. (2003) Biophys J 85, 1560-75; Siskind, L. J., Kolesnick, R. N. and Colombini, M. (2002) J Biol Chem 277, 26796-803; Kuwana, T., Mackey, M. R., Perkins, G., Ellisman, M. H., Latterich, M., Schneiter, R., Green, D. R. and Newmeyer, D. D. (2002) Cell 111, 331-42) and the molecular identity of MAC is not known. These agents may modify as yet unidentified lipid components of the MAC. In fact, the results of Polster et al. suggest that dibucaine and propranolol inhibit Bax-induced permeability changes through a direct interaction with the lipid membrane (Polster, B. M., Basanez, G., Young, M., Suzuki, M. and Fiskum, G. (2003) J Neurosci 23, 2735-43). Finally, dibucaine and trifluoperazine inhibit phospholipase A2 (Broekemeier, K. M., Schmid, P. C., Schmid, H. H. and Pfeiffer, D. R. (1985) J Biol Chem 260, 105-13), which again may implicate a role for lipids in MAC activity. However, it is unlikely there is functional phospholipase A2 in the reconstituted system used in this study.

Trifluoperazine, dibucaine and propranolol reduce the conductance of MAC below 1.3 nS. It will be important to compare these agents with those identified as blockers of Bax channels (Bombrun, A., Gerber, P., Casi, G., Terradillos, O., Antonsson, B. and Halazy, S. (2003) J Med Chem 46, 4365-8; Polster, B. M., Basanez, G., Young, M., Suzuki, M. and Fiskum, G. (2003) J Neurosci 23, 2735-43), as Bax may be a component of MAC. Our previous studies indicate cytochrome c does not affect MAC if the conductance is below 1.9 nS (Guo, L., Pietkiewicz, D., Pavlov, E. V., Kasianowicz, J. J., Korsmeyer, S. J., Antonsson, B. and Kinnally, K. W. (2004) American Journal of Physiology, Cell Biology. 286, C1109-17), i.e., cytochrome c likely does not transit a pore with a conductance below 1.9 nS. Hence trifluoperazine, dibucaine, and propranolol effectively eliminate the MAC's permeability for cytochrome c and should short circuit the death cascade. Future studies will include examination of the effect of other pharmacological agents that modify MAC on the progression of apoptosis in vitro and in vivo. Ultimately, other agents may be identified that reduce the volume of cell death associated with, e.g., stroke and myocardial infarction.

### Example 2

### The Spinal Cord Injury Model to Assess the Compounds of the Invention in vivo

One means of assessing the compounds of the invention in neuronal injury or in neurodegenerative diseases is by use of the spinal cord injury model. The NYU Impactor and rat contusion models of spinal cord injury have been extensively described. (Kwo S, Young W, DeCrescito V (1989) Journal of Neurotrauma 6: 13-24; Huang P, Young W (1994). Journal of Neurotrauma 11: 547-62; Basso M, Beattie M, Bresnahan J, Anderson D K, Faden A, et al. (1996) Journal of Neurotrauma l in press; Constantini S, Young W (1994) Journal of Neurosurgery 80: 97-111). Rats are anesthetized with an intraperitoneal dose of pentobarbital (40 mg/kg female, 60 mg/kg male), suspended with clamps placed on the T8 and T11 dorsal processes, and injured at one hour after induction of anesthesia. The impactor drops a 10 g rod a distance of 12.5, 25.0, or 50.0 mm onto the dorsal surface of T9-10 spinal cord, exposed by laminectomy. Two digital optical potentiometers measure the trajectory of the falling rod and vertebral movement with a precision of ±20 μm and ±20 μsec. Cord compression rate (Cr) is calculated from the distance of cord compression divided by time required for compression. Cr correlates linearly with 24-hour lesion volumes (r<0.900), as well as 6-week locomotor scores and spared white matter (WM).

The rats receive daily Keflin® (15 mg/kg b.i.d. subcutaneous) for one week after injury and then Baytril® (5 mg/kg b.i.d. subcutaneous) for 10 days for recurrent urinary tract infections. All rats receive twice daily bladder compression until they recover voiding. The rats are assessed weekly for locomotor recovery, using the Basso-Beattie-Bresnahan (BBB) scale developed at Ohio State University. At 11 weeks after injury, the rats receive injections of BDA (Biotinylated dextran amine) into their motor cortex to label their corticospinal tracts. At 12 weeks, the rats are anesthetized with pentobarbital and perfused with formaldehyde (4%) solution through the heart.

Locomotor Scoring

The BBB score is a 21-point scale representing 21 stages of locomotor recovery after spinal cord injury. The scale is based on unique combinations of scored behaviors, ranked according to time of appearance after injury. Behaviors that appear last or in the least severely contused rats have higher scores than behaviors that appear early on or in more severely injured rats. The score can be divided into three parts: from 0-8, the scores emphasize voluntary movements of hindlimb joints; from 8-14, the scores represent standing and stepping with progressively better forelimb_hindlimb coordination; from 15-21, the scores indicate greater strength and better foot placement and balance. Each score represents a unique combination of behaviors, providing a non-ambiguous ordinal scale. The scale is described in Table 1.

Every week, the rats are placed in a standard open field (a plastic tub with walls) and are observed by two trained investigators from two sides for 4 minutes. Characteristics of locomotion are checked off on a scoring sheet and the final score represents the consensus opinion of the two observers. Detailed inter-rater reliability analyses indicate that experienced observers can achieve a standard deviation of less than 1 point on the scale. All scoring is done by people who are not aware of the treatments received by individual rats. Treatments are masked through the analysis except for an interim analysis at 6 weeks to perform a “futility” test to determine whether there is sufficient difference among treatment groups to continue the trial.

TABLE 1

| BBB scores | | Comments |
|---|---|---|
| 0 | No observable hindlimb (HL) movement | None |
| 1 | Slight movement on one or two HL joints | Slight ≦50% of joint range |
| 2 | Extensive movement of one HL joint and | Extensive ≧50% of joint range |
| | slight movement of the other joint | |
| 3 | Extensive movement of two HL joints | Two joints = usually hip & knee |
| 4 | Slight movement of three HL joints | Three joints = hip, knee & ankle |
| 5 | Slight movement of two HL joints & | |
| | extensive movement of third HL joint | |
| 6 | Extensive movement of two joints HL | Third joint = usually the ankle |
| | joints & slight movement of third HL joint | |
| 7 | Extensive movement of all three HL joints | |
| 8 | Sweeping with no weight support or | Sweeping = rhythmic 3 joint movement |
| | Plantar placement with no weight support | |
| 9 | Plantar placement with weight support OR | Weight support = HL extensor contraction with elevation of |
| | Dorsal stepping with weight support | hindquarters in stance |
| 10 | Occasional weight supported steps with no | Occasional = >5% & ≦50% |
| | forelimb-hindlimb (FL-HL) coordination | Steps = plantar steps with weight support |
| 11 | Frequent to consistent steps (FCS) with no | Frequent = 51-94% of the time |
| | coordination | Consistent = 95-100% of the time |
| 12 | FCS with occasional coordination | 6-50% bouts of locomotion coordinated |
| 13 | FCS with frequent coordination | 51-95% bouts of locomotion coordinated |
| 14 | Consistent coordinated steps (CCS) & | Rotated = internal or external rotation |
| | paw rotated on placement & liftoff OR | |
| | Frequent steps, consistent coordination | |
| | With occasional dorsal steps | |
| 15 | CCS & no or occasional toe clearance & | Parallel = paw placement to body |
| | parallel paw position on initial placement | Toe clearance = steps without toe drag |
| 16 | CCS & frequent toe clearance | Frequent toe clearance ≧50% no toe drag |
| 17 | CCS & parallel paw on placement and | |
| | liftoff | |
| 18 | CCS & consistent toe clearance | Consistent toe clearance ≦4 toe drags |
| 19 | CCS & parallel paw on placement and | Tail down = touches ground when walking |
| | liftoff | |
| | Tail down part or all the time | |
| 20 | CCS & parallel paw on placement and | Trunk instability = lateral weight shifts, waddling, lurching |
| | liftoff | |
| | Tail consistently up, trunk unstable | |
| 21 | CCS, consistent toe clearance, parallel | Consistent trunk stability no lurching |
| | paws, tail consistent up, consistent trunk | |
| | stability | |

Histological Assessment of Injured Spinal Cords

The spinal cords are removed and immersed in 4% formaldehyde for several days. A 1-mm section at the contusion center is removed and embedded in plastic for analysis of spared white matter. The cord sections are stained with toluidine blue to show axons and myelin.

White matter sparing is estimated from the cross-sectional areas of spinal cord that contain myelinated axons. Percent (WM) white matter sparing is calculated by dividing the area of WM sparing by the total cross-sectional area.

The proximal and distal pieces of cord are passed through 50% and 75% sucrose solutions, and frozen at −5° C. The proximal cords are horizontally frozen-sectioned at 40 μm thickness to show the labelled corticospinal tract. This distal cords are coronally frozen-sectioned and every fifth section is reacted with DAB and nickel (0.25 mg/ml diaminobenzidine +0.04% nickel) to stain labelled corticospinal tract. The sections are observed with a dark-field condenser.

### Example 3

### Prevention of Neuronal Cell Death by the Compounds of the Present Invention

The following tests may also be carried out to test the effects of the compounds of the present invention on neuronal cell protection and correspondingly, are capable of preventing apoptosis.

Apoptosis, also known as programmed cell death, plays a key role in the normal development of the nervous system. Apoptosis leads to elimination of up to 50% of developing neurons, and is the mechanism responsible for matching neuronal populations to target size (Oppenheim, R. W. (1991). Survival is largely controlled by a limiting supply of target-derived growth factors, but is further influenced by afferent stimulation (Linden, R. (1994)). Accumulating evidence suggests that apoptosis is also involved in pathological neuronal death, which occurs in neurodegenerative disorders such as Alzheimer disease (Bredesen, O. E. (1996)).

Cell Cultures

Cerebellar granule neurons are prepared from 6-8 day-old mice. The cells are re-suspended and cultured in X1 medium (BME basal medium (Life Technologies) with 1 mg/ml BSA, 2.2 mg/ml NaHCO3, 100 μg/ml transferrin, 10 μg/ml insulin, 4 nM thyroxine 30 nM NaSeO3, 0.027 TIU/ml aprotinin, 5 IU/ml penicillin and 5 μg/ml streptomycin).

Hippocampal neurons are prepared from 18-day rat embryos as described (Lochter, A., et al (1991); Brewer, G. J., et al (1993)). Neurons are re-suspended and plated in serum-free Dulbecco's modified Eagle's medium/Nutrient Mix F12 (Life Technologies, Inc.) supplemented with N2 components, 33 mM D_glucose, 1 mM pyruvate, 1 mg/ml BSA, and 5 mM Hepes.

Cerebellar Granule Cells Maintained in Serum-Free Medium Undergo Programmed Cell Death

When cerebellar granule cells are cultured in serum-free medium at high densities (2.5_{—}3.0×10⁵ cells/cm²), less than 10% of the seeded cells survive after two weeks. To test whether the cell death occurring in these cultures could be attributed to apoptosis or necrosis, one can measure condensation of chromatin and fragmentation of nuclei, which are typical features of apoptotic cells (Raff, M. C. (1992); Ellis, R., et al (1991)). Upon staining of cerebellar granule cells with the dye Hoechst 33258, nuclear condensation can be observed. Moreover, DNA can be degraded, with cleavage of chromatin into nucleosomal fragments. Further testing for apoptosis can be done by measuring Annexin V. Morphological features of the cerebellar granule cells after 5 days in culture with or without the addition of the compounds of the invention is also monitored. The cerebellar granule cells may be cultured in the absence of serum, but in the presence of the compounds of the invention or in a vehicle control, and cell samples may be monitored for apoptosis at various intervals to determine the effect of the compounds of the invention on apoptosis.

Cell Survival Assay

Mitochondrial function is assessed as a measure of cell viability, by measuring the conversion of soluble MTT (3-[4,5-dimethylthiazol-2-yl]2,5-diphenyltetrazolium bromide) into an insoluble dark blue formazan reaction product, using the procedure of Mosmann (Mosmann, t. (1983)) modified according to the instruction manual for the MTT Cell Proliferation Kit (Boehringer Mannheim). Cerebellar granule cells or hippocampal neurons are seeded in 96-well plates at densities of 1×10⁵ or 5×10⁴ cells/well, respectively. The percentage survival determined by the MTT assay is nearly identical to that determined by trypan blue exclusion staining. Values are expressed as the % of control wells (with PLL only) in each experiment.

Viability is also assessed by differential staining of live and dead cells using an assay kit (“LIVE/DEAD® Viability/Cytotoxicity assay Kit^{m}; Molecular Probes, Inc., Eugene, Oreg.) according to the manufacturers instructions. Fragmentation of DNA is analyzed as described (Hockenbery, D., et al (1990); D'Mello, et al (1993); Yang, R. J., et al (1996)). Cells may be cultured in the absence or presence of the compounds of the invention or in a vehicle control, and cell samples may be monitored for viability at various intervals to determine the effect of the compounds of the invention on cell viability.

### Example 4

### Traumatic Brain Injury Model for Testing the Compounds of the Invention

The compounds of the invention can be tested for efficacy in an animal model of traumatic brain injury. Specifically, compounds can be evaluated in the lateral fluid percussion injury (FPI) model developed by McIntosh (McIntosh T. K., et. al (1989), Neuroscience 28:233-244). This model replicates many of the pathological aspects found in humans who have sustained traumatic brain injury. Furthermore, the damage caused by the injury is sensitive to pharmacological intervention, and thus provides a robust method for preclinical efficacy testing. In this model, animals are subjected to a fluid percussion brain injury, and are then given the test compounds shortly thereafter. At specific time points, animals are evaluated using outcome measures that assess cognitive and locomoter deficits that are consequences of the injury. In addition, following behavioral analysis, animals are sacrificed and their brains assessed with respect to a variety of histopathological parameters. A comparison is then made between compound-treated and untreated animals to determine if the compound can attenuate the behavioral deficits and histological derangements normally caused by Fluid Percussion Injury.

Experimental Protocol

For these studies, male rats (350-400 g) are used for injuries. All animals are injured at a moderate/severe injury level (2.5-3.0 atm) via a craniotomy positioned over the left parietal cortex centered between Bregma and Lambda. Fifteen minutes after injury, they are randomly assigned into groups and treated with either a compound of the invention or vehicle. A dose response study may be done to determine optimal levels of drug needed for efficacy. Compound or vehicle are delivered via IP injection 15 minutes after injury, followed by oral gavage or IP injection b.i.d. for up to 5 to 7 days following the injury. Animals are assigned to one of 2 study groups. Animals in the first group are tested for memory deficits 48 hrs post injury, and then sacrificed for acute analysis of Edema (Brain water content). Animals in the second group are tested for locomotor deficits and learning deficits at a later time point. Following behavioral testing, these animals are sacrificed, and their brains are analyzed for tissue damage (cortical volume loss, and neuronal cell counts), and other histopathological readouts.

Analysis of Post-Trauma Cognitive Deficits And Brain Water Content

Cognitive Deficits

The ability of a test compound to restore cognitive deficits in spatial memory and learning is evaluated at 48 hrs post injury using the Morris watermaze paradigm (Smith D. H. et al. (1991), J. Neurotrauma 8(4): 259-269; Schmidt, R. H. et al. (1999), J. Neurotrauma 16(12): 1139-1147). In this assay, animals are trained to swim in a 1 m or 2 m circular water pool containing a submerged platform. Animals learn to orient themselves in the pool and to locate the platform by using distant visual cues placed on the walls of the room. In the learning paradigm, the animals are trained to find the platform, and the reduction in latency with increasing number of training sessions is recorded. In the memory paradigm, animals are given 20 training runs over a 2 day period prior to the injury. Following the second training session, the injury is delivered. 48 hours after injury, animals are tested for memory function by placing them into the pool with the platform removed, and recording the time spent swimming in defined areas of the pool. A significant memory score is generated if the animal spends more time swimming in areas where the platform was or close to where the platform was than in the other areas of the pool.

To analyze a test compound, animals are tested in the memory paradigm. Animals from test-compound treated and vehicle treatment groups are trained to swim to the submerged platform over a 2 day period (10 swims/day). Following training, animals are injured, and treated with a test compound via IP injection 15 minutes following injury. For the following 48 hours, they receive the test compound twice daily via oral gavage or IP injection. 48 hrs following injury, they are tested in the memory paradigm.

This assay is a very useful test of cognitive function because it is sensitive to degree of injury, and is responsive to pharmacological interventions.

Brain Water Content

Following water maze analysis, animals are euthanized, and their brains dissected. Brains are chilled on a frozen block, and a 3-5 mm coronal section surrounding the injury site is dissected. This section is subdissected into the following regions: left parietal cortex (injury region), contralateral parietal cortex (control), parietal cortex adjacent to the injury region (left and right), and left and right hippocampal regions. Tissue pieces are weighed, and then dried overnight at 100° C.

Cerebral edema is the percent of tissue weight contributed by water, and is calculated by the formula:
% water=(wet weight−dry weight)/(wet weight)*100

In this manner, the ability of the compounds used in the invention to attenuate posttraumatic cerebral edema is evaluated.

Analysis of Post-Trauma Neurological Deficits and Histological

Neurological Evaluation

Neurological testing is used to assess the ability of the compounds of the invention, to attenuate the locomoter deficits normally induced by the Fluid Percussion Injury. Testing is begun 24 hr after the injury, and continues weekly for 1-4 weeks depending on the outcomes observed. Locomoter analysis is based on a set of tests that primarily assess locomoter and vestibulomotor function. This analysis includes a composite neuroscore, that largely involve tests of reflexive locomoter function. Additional tests include the beam balance (Scherbel, U. et al. (1999), Proc. Natl. Acad. Sci. 96: 8721-8726) and the rotating pole (Mattiasson, G. V. et al. (2000), J. Neuroscience Methods 95: 75-82), which test coordination and vestibulomotor function. The composite neuroscore evaluates the following behaviors: 1) contralateral forelimb flexion upon suspension by the tail; 2) hindlimb flexion upon suspension by the tail; 3) resistance to lateral pulsion to the left and to the right; 4) Ability to stand on an inclined angle board. Animals are evaluated on their ability to execute the appropriate movements as well as their strength and coordination. They are graded on a 5 point scale with Grade 4 representing normal behavior and Grade 0 representing a severe deficit. The combined neuroscore is the sum of scores from the 4 tests.

In addition to the neuroscore, vestibulomoter function is evaluated using the rotating pole and the balance beam. On the balance beam, animals are trained to traverse a 2 cm wooden beam. After training, they are placed on the beam and tested. They are evaluated for ability to traverse the beam in a normal fashion, coordination of movements, and foot slips, and as in the neuroscore, are given a score from 0-4 depending on the degree of deficit. The rotating pole is an approximately 2 m pole that can rotate in either a clockwise or counterclockwise direction. Prior to evaluation, the animal is trained to traverse the non-rotating pole from one end to other. For testing, the pole will be set to rotate at a constant speed, and the animal is then placed onto the pole. Latency to traverse the pole, as well as foot slips and/or falls is evaluated. Animals are scored on a 5 point scale with 0 indicating the greatest deficit.

Taken together, these scores of locomoter activity provide a sensitive and accurate assessment of severity of injury.

Histopathological Analysis

Subsequent to neurological analysis, brain tissue from the animals is prepared for histological analysis. Animals are euthanized, and then transcardially perfused with 4% Paraformaldehyde (PFA). Their brains are dissected, and post fixed in 4% PFA, cryoprotected in 30% sucrose, and then frozen for sectioning. Serial coronal sections from the injury region are cut and stained with Hematoxylin and Eosin (H&E) or 5% cresyl violet (Nissl). H&E sections are used to evaluate lesion volume, while Nissl is used to assess cell loss due to injury. In each case, the contralateral side serves as a control for the injured side of the brain.

For contusion volume analysis, a single section taken every millimeter from 1.3 mm to 6.3 mm posterior to Bregma is examined under low magnification. Image analysis software (e.g. MCID/M4 image software, or NIH image) may be used to capture the images, and to calculate hemispheric area of the ipsilateral and contralateral side of each section. The volume of the ipsilateral and contralateral hemispheres is then computed by integrating the area of each section and the distance between sections.

To assess cell loss following injury, cell counts are performed in the CA3 hippocampal region. Nissl stained sections are examined at moderate magnification, and cells with neuronal morphology in the CA3 region along an arc of defined length are counted. The number of cells obtained is compared to the number of neurons counted along a similar arc in CA3 of the contralateral (uninjured) side to determine the degree of cell loss on the injured side.

Data Analysis

For analysis of continuous variables compared across groups, (e.g. watermaze latency) data is examined using analysis of variance (ANOVA) followed by post-hoc Newman-Keuls tests. Ordinal measurements such as combined neuroscores are analyzed using the non-parametric Kruskal-Wallis ANOVA followed by post-hoc non-parametric Mann-Whitney U-tests.

1. A method of inducing apoptosis in cells in vitro or in vivo comprising administering an agent that promotes opening of the mitochondrial apoptosis induced channel (MAC), wherein said opening results in release of cytochrome c and subsequent release of a death signal.

2. The method of claim 1, wherein said death signal is not cytochrome c, but is a small molecule other than cytochrome c capable of traversing gap junctions.

3. The method of claim 1, wherein said channel is integral to the bystander effect in vivo and wherein said release of a death signal results in apoptosis of cells outside of the area of the immediate cellular or tissue insult.

4. A method of treating a disease or condition characterized in part by the presence of apoptotic cell death, comprising administering an agent that prevents opening of the mitochondrial apoptosis-induced channel (MAC), or promotes closure of the mitochondrial apoptosis-induced channel (MAC).

5. The method of claim 4, wherein said disease or condition is selected from the group consisting of stroke, myocardial infarction, Alzheimer's disease, traumatic brain injury, spinal cord injury, AIDS and any other medical condition characterized in part by the presence of apoptotic cell death.

6. The method of claim 4, wherein the agent is selected from the group consisting of trifluoperazine, dibucaine and propranolol.

7. A method of treating a disease or condition wherein said disease or condition is characterized by unwanted or undesirable cellular proliferation, comprising administering an agent that promotes opening of the mitochondrial apoptosis-induced channel (MAC) and apoptosis.

8. The method of claim 7, wherein said opening results in release of cytochrome c and subsequent release of a death signal.

9. The method of claim 8, wherein said death signal is not cytochrome c, but is a small molecule other than cytochrome c capable of traversing gap junctions.

10. The method of any of claims 7, 8, or 9, wherein said disease or condition is selected from the group consisting of a cancer and any other hyperproliferative disorder for which inhibition of cellular proliferation and cell death is desirable.

11. The method of claim 10, wherein cell death is achieved by apoptosis.

12. A pharmaceutical composition comprising an agent that modulates MAC and a pharmaceutically acceptable carrier.

13. The composition of claim 12, wherein said agent is selected from the group consisting of an agent that promotes opening of MAC, an agent that promotes closing of MAC, and an agent that inhibits the opening of MAC, and a pharmaceutically acceptable carrier.

14. A method for identifying modulators of a mitochondrial apoptosis induced channel (MAC), comprising contacting a test compound with said channel and detecting the activity of said channel.

15. The method of claim 14, wherein said activity, when inhibited, identifies a modulator useful for treatment of a disease or condition selected from the group consisting of stroke, Alzheimer's disease, myocardial infarction, traumatic brain injury and spinal cord injury.

16. The method of claim 14, wherein said activity when enhanced identifies a modulator useful for treatment of a disease or condition selected from the group consisting of a cancer and any hyperproliferative disorder for which induction of apoptosis is desirable.
